SEQUENCE LISTING (7 sequences)

SEQ ID NO: 1 - DNA, 765 bp, Homo sapiens
  source 1..765: mol_type = unassigned DNA
atgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggctaccaggtgtccttctgcctcgtgatggtgctgctgttcgccgtggacaccggcctgtacttcagcgtgaaaacaaacatcagaagcagcacccgggactggaaggaccacaagttcaagtggcggaaggacccccaggacaagtga

SEQ ID NO: 2 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 3 - DNA, 483 bp, Homo sapiens
  source 1..483: mol_type = unassigned DNA
atgtaccggatgcagctgctgagctgtatcgccctgtctctggccctcgtgaccaacagcgcccctaccagcagcagcaccaagaaaacccagctgcagctggaacatctgctgctggacctgcagatgatcctgaacggcatcaacaactacaagaaccccaagctgacccggatgctgaccttcaagttctacatgcccaagaaggccaccgaactgaaacatctgcagtgcctggaagaggaactgaagcccctggaagaagtgctgaacctggcccagagcaagaacttccacctgaggcccagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgaccggctccgagaaggacgagctgtga

SEQ ID NO: 4 - protein, 160 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSEKDEL

SEQ ID NO: 5 - protein, 405 aa, synthetic construct
  REGION 1..405: note = Description of Artificial Sequence: Synthetic polypeptide
MDWIWRILFLVGAATGAHSAQPANIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKGGGGSGGGGSGGGGSGGGGSEVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSSAAALSNSIMYFSHFVPVFLPAKPTTTPAPRPPTPAPTIASQPLSLRPEACRPAAGGAVHTRGLDFACFWVLVVVGGVLACYSLLVTVAFIIFWVRLKIQVRKAAITSYEKSDGVYTGLSTRNQETYETLKHEKPPQ

SEQ ID NO: 6 - protein, 407 aa, synthetic construct
  REGION 1..407: note = Description of Artificial Sequence: Synthetic polypeptide
MDWIWRILFLVGAATGAHSAQPADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSAAALSNSIMYFSHFVPVFLPAKPTTTPAPRPPTPAPTIASQPLSLRPEACRPAAGGAVHTRGLDFACFWVLVVVGGVLACYSLLVTVAFIIFWVRLKIQVRKAAITSYEKSDGVYTGLSTRNQETYETLKHEKPPQ

SEQ ID NO: 7 - protein, 275 aa, synthetic construct
  REGION 1..275: note = Description of Artificial Sequence: Synthetic polypeptide
MDWIWRILFLVGAATGAHSAQPADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKSSGGGGSGGGGSGGGGSGGGGSGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

(19) United States
(12) Patent Application Publication
(10) Pub. No.: US 2026/0275301 A1
(43) Pub. Date: Sep. 17, 2026

(54) Methods for Preparing and Storing Immortalized Cytolytic Natural Killer Cells

(71) Applicant: ImmunityBio, Inc., San Diego, CA (US)

(72) Inventors: Shannyn Bessette, San Diego, CA (US); Syed Raza Ali, San Diego, CA (US); Manju Saxena, San Diego, CA (US)

(21) Appl. No.: 19/555,083

(22) Filed: Mar. 3, 2026

Related U.S. Application Data
(63) Continuation-in-part of application No. 17/260,545, filed on Jan. 14, 2021, now abandoned.
(63) Continuation-in-part of application No. 16/964,962, filed on Jul. 24, 2020, now abandoned.
(60) Provisional application No. 62/624,624, filed on Jan. 31, 2018.
(60) Provisional application No. 62/696,143, filed on Jul. 10, 2018.

(51) Int. Cl. C12N 5/00; C12N 5/09

(52) U.S. Cl. CPC C12N 5/526; C12N 5/0693; C12N 2510/04

(57) ABSTRACT

Provided herein are methods of preparing and storing NK-92® cells. The methods comprise storing and freezing NK-92® cells in freezing medium comprising a first composition and a second composition. Also disclosed are methods of harvesting NK-92® cells comprising collecting the cells from a cell culture and washing the collected cells in a buffer comprising albumin.

### CROSS-REFERENCE TO RELATED APPLICATIONS

This application is a continuation-in-part of U.S. application Ser. No. 17/260,545, filed on Jan. 14, 2021, and U.S. application Ser. No. 16/964,962, filed Jul. 24, 2020, which claim priority to PCT/US2019/041028, filed Jul. 9, 2019, and PCT/US2019/015841, filed Jan. 30, 2019, respectively; which claim priority to U.S. Provisional Application No. 62/696,143 filed on Jul. 10, 2018, and U.S. Provisional Application No. 62/624,624, filed on Jan. 31, 2018, respectively; the contents of which are herein incorporated by reference in their entireties for all purposes.

### SEQUENCE LISTING IN ELECTRONIC FORMAT

This application contains an Electronic Sequence Listing submitted as an XML file entitled 104066-1544962-8920US_SL.xml, which is 11,066 bytes in size, and which was created on Mar. 13, 2026, the contents of which are hereby incorporated by reference in its entirety.

### BACKGROUND

Natural killer (NK) cells are cytotoxic lymphocytes that constitute a major component of the innate immune system. NK cells, generally representing about 10-15% of circulating lymphocytes, bind and kill targeted cells, including virus-infected cells and many malignant cells, non-specifically with regard to antigen and without prior immune sensitization. Herberman et al., Science 214:24 (1981). Killing of targeted cells occurs by inducing cell lysis. NK cells used for this purpose are isolated from the peripheral blood lymphocyte (“PBL”) fraction of blood from the subject, expanded in cell culture in order to obtain sufficient numbers of cells, and then re-infused into the subject. NK cells have been shown to be somewhat effective in both ex vivo therapy and in vivo treatment. However, such therapy is complicated by the fact that not all NK cells are cytolytic and the therapy is specific to the treated patient.

NK-92® cells have previously been evaluated as a therapeutic agent in the treatment of certain cancers. Unlike NK cells, NK-92® is a cytolytic cancer cell line which was discovered in the blood of a subject suffering from a non-Hodgkins lymphoma and then immortalized ex vivo. NK-92® cells lack the major inhibitory receptors that are displayed by normal NK cells, but retain the majority of the activating receptors. NK-92® cells do not, however, attack normal cells nor do they elicit an unacceptable immune rejection response in humans. Characterization of the NK-92® cell line is disclosed, e.g., in WO 1998/49268 and U.S. Pat. No. 8,034,332.

However, the poor efficiency of cell harvesting remains a significant challenge for producing sufficient NK-92® cells for various therapeutic applications. Conventional harvesting procedures typically involve collecting cells from cell culture medium and washing the cells in buffer such as PBS. The multiple washes in PBS cause cell loss and a significant reduction in viability. In some instances, cells are washed in a medium, such as X-VIVO™10; this is also not ideal because the final product formulation requires two additional steps including centrifugation for removal of X-VIVO™M10. These steps increase processing time and cause cell stress and cell loss due to the repeated centrifugation steps that are required.

Further, the storage of the NK-92® cells remains a challenge. For storage, NK-92® cells are typically stored in freezing media and thawed before use. The freeze-thaw procedures induce molecular cell stress responses, causing apoptosis and/or necrosis upon; and the formation of intracellular ice can have significant negative effect on cell viability. As a result, the cell viability is often low, which impairs the therapeutic effect of the NK-92® cells. Further, the existing, routine freezing medium uses a high concentration of DMSO, typically 10%, which can also decrease viability of the cells. In addition, many of these traditional cell freezing medium comprises components that are not suitable for direct infusion to patients. After thawing, the cells must be cultured and reformulated in a medium that is suitable for infusion. This requisite reformulation step would increase the cost for the therapy and also, could introduce human operational error. Thus, the needs remain for an economical, cell freezing medium that can be used to store NK-92® cells with miminized damages to cells, and the NK-92® cells after thawing can be directly administered to patients.

### SUMMARY

Provided herein are methods for preparing and storing NK-92® cells.

Provided herein are methods for storing NK-92® cells in a cell freezing medium and compositions comprising NK-92® cells and the cell freezing medium.

Provided herein is a method of freezing NK-92® cells, the method comprising: contacting NK-92® cells with a cell freezing medium, wherein the cell freezing medium comprises a first composition and a second composition, wherein the first composition comprises: (a) one or more electrolytes selected from the group consisting of potassium ions, sodium ions, magnesium ions, and calcium ions; (b) one or more macromolecular agents selected from the group consisting of human albumin, polysaccharide and colloidal starch; (c) a pH buffer; (d) at least one sugar; (e) at least one sugar alcohol; (f) an impermeant agent selected from the group consisting of lactobionate, gluconate, citrate and glycerophosphate; and (g) DMSO; and wherein the second composition comprises albumin.

Optionally, the cells are contacted with the second composition before adding the first composition.

Optionally, the sugar alcohol is a 6-carbon sugar alcohol. Optionally, the sugar is a simple sugar. Optionally, the simple sugar is selected from the group consisting of glucose, dextran, dextrose, trehalose, and maltose.

Optionally, the albumin in the second composition is human albumin. Optionally, the second composition comprises 2-10% human albumin.

Optionally, the first and second composition are mixed at a volume ratio that ranges from 1:5 to 5:1, for example, about 1:1. Optionally, the first composition comprises 10% DMSO. Optionally, the first composition further comprises one or more substrates effective for regeneration of ATP. Optionally, the one or more substrates are selected from the group consisting of adenosine, fructose, ribose and adenine.

Optionally, the first composition further comprises glutathione. Optionally, the first composition further comprises one or more impermeant agents that can maintain ionic and osmotic balance during hypothermia.

Optionally, the cell freezing medium comprises less than 10% DMSO.

Optionally, the method further comprises freezing the cells using a controlled rate freeze to reach a final temperature of −80° C. or lower. Optionally, the method further comprises storing the cells at −80° C. to −196° C.

Optionally, the method further comprises thawing the preserved cells. Optionally, the cells are thawed at 37° C. Optionally, more than 70% of the cells are viable at the time of thawing. Optionally, more than 90% of the cells are viable at the time of thawing.

Optionally, the thawed cells have a direct cytoxicity of at least 80% against K562 cells at an effector to target ratio of 10:1. Optionally, the thawed cells have a direct cytoxicity that is 70-100% of that of the NK-92® cells before the cells are frozen. Optionally, the thawed cells have a ADCC toxicity of at least 80-120% against Ramos cells at an effector to target ratio of 10:1, in the presence of an antibody targeting the Ramos cells. Optionally, the thawed cells have a ADCC cytoxicity that is 80-100% of that of the cells before the cells are frozen.

Optionally, the method further comprises administering the thawed cells to a patient in need via infusion.

Optionally, the NK-92® cells express a cytokine, Fc Receptor, chimeric antigen receptor, or a combination thereof.

Also provided is a NK-92® cell culture comprising: NK-92® cells and a cell freezing medium, wherein the cell freezing medium comprises a first composition and a second composition, wherein the first composition comprises: (a) one or more electrolytes selected from the group consisting of potassium ions, sodium ions, magnesium ions, and calcium ions; (b) one or more macromolecular agents selected from the group consisting of human albumin, polysaccharide and colloidal starch; (c) a pH buffer; (d) at least one sugar; (e) at least one sugar alcohol; (f) an impermeant agent being at least one member selected from the group consisting of lactobionate, gluconate, citrate and glycerophosphate; and (g) DMSO; and wherein the second composition comprises albumin. Optionally, the sugar alcohol is a 6-carbon sugar alcohol. Optionally, the simple sugar is selected from the group consisting of glucose, dextran, dextrose, trehalose, and maltose. Optionally, the second composition is human albumin. Optionally, the second composition is 2-10% human albumin.

Optionally, the first composition and second composition is mixed at a volume ratio ranging from 1:5 to 5:1, for example, about 1:1. Optionally, the first composition comprises 10% DMSO. Optionally, the first composition further comprises a substrate effective for the regeneration of ATP.

Optionally, the substrate in the first composition is selected from the group consisting of adenosine, fructose, ribose and adenine. Optionally, the first composition comprises 20-60 mM potassium ions. Optionally, the first composition comprises 50-150 mM sodium ions. Optionally, the first composition comprises 0.001-1 mM magnesium ions. Optionally, the first composition further comprises glutathione. Optionally, the cell culture is kept at 80° C. to −196° C. Optionally, at least 70% of NK-92® cells in the cell culture that has been kept at −80° C. to −196° C. are viable after cell culture is thawed.

Optionally, the NK-92® cells disclosed comprises a cytokine, Fc Receptor, chimeric antigen receptor, or a combination thereof.

Also provided is a cell freezing medium produced by mixing a first composition and a second composition at a one to one ratio, wherein the first composition comprises: (a) one or more electrolytes selected from the group consisting of potassium ions, sodium ions, magnesium ions, and calcium ions; (b) one or more macromolecular agents selected from the group consisting of human albumin, polysaccharide and colloidal starch; (c) a pH buffer; (d) at least one sugar; (e) at least one sugar alcohol; (f) an impermeant agent selected from the group consisting of lactobionate, gluconate, citrate and glycerophosphate; and (g) DMSO; and wherein the second composition comprises albumin.

Provided herein are methods of harvesting NK-92® cells comprising collecting NK-92® cells from a cell culture and washing the collected NK-92® cells by a buffer comprising 1-5% albumin. The NK-92® cells may be those modified to express one or more transgenes, for example, the NK-92® cells can be modified to express a cytokine, a Fc receptor, a chimeric antigen receptor, or a combination thereof.

Optionally, the collecting NK-92® cells comprising centrifuging the NK-92® cells in the cell culture. Optionally, the method further comprise placing the washed NK-92® cells in a infusion bag. Optionally, the wash is performed by centrifuging the cells and then resuspending the cells in the wash buffer. Optionally, the wash is performed at least three times, e.g., four to six times. Optionally, the method recovers at least 80% of the NK-92® cells. Optionally, the viability of the harvested cells is at least 90%.

Optionally, the NK-92® cells that have been harvested have substantially the same cytotoxicity and/or viability as control NK-92® cells that have not been harvested. Optionally, the NK-92® cells that have been harvested have substantially the same cytotoxicity and/or viability as the NK-92® cells before harvesting. Optionally, the NK-92® cells that have been harvested have a cytotoxicity of 80-100% on K562 cells. Optionally, the buffer contains 2-5% albumin, e.g., 3-5% albumin, or 5% albumin. Optionally, the buffer lacks sugar. Optionally, the buffer lacks dextran. Optionally, the centrifugation is by continuous centrifugation. Optionally, the albumin is human plasma albumin or human serum albumin. Optionally, the NK-92® cells express a cytokine, Fc Receptor, a chimeric antigen receptor, or a combination thereof.

The foregoing general description and the following detailed description are exemplary and explanatory and are intended to provide further explanation of the disclosure. Other objects, advantages and novel features will be readily apparent to those skilled in the art.

### BRIEF DESCRIPTION OF THE DRAWINGS

The objects, features and advantages will be more readily appreciated upon reference to the following disclosure when considered in conjunction with the accompanying drawings.

FIG. 1 is a schematic illustration of an exemplary process of harvesting NK-92® cells.

### DETAILED DESCRIPTION

Provided herein are methods of preparing NK-92® cells for storage, for example, by cryopreservation. The methods provide the benefit of maintaining cell morphology and function of the NK-92® cells. Also provided herein are methods of harvesting NK-92® cells, for example, from frozen stocks, using a buffer containing 1-5% albumin, optionally 5% human albumin. After washing, the cells can be directly used for therapeutic applications, such as infusion, without the need for further processing steps or formulation. The methods disclosed herein advantageously reduce processing complexity and processing times, and minimize cell loss and cell stress.

After reading this description, it will become apparent to one skilled in the art how to implement various alternative embodiments and alternative applications. However, not all embodiments are described herein. It will be understood that the embodiments presented here are presented by way of an example only, and not limitation. As such, this detailed description of various alternative embodiments should not be construed to limit the scope or breadth of the disclosure as set forth herein. It is to be understood that the aspects described below are not limited to specific compositions, methods of preparing such compositions, or uses thereof as such may, of course, vary.

### Terminology

Unless defined otherwise, all technical and scientific terms used herein have the same meaning as commonly understood by one of ordinary skill in the art.

In this specification and in the claims that follow, reference will be made to a number of terms that shall be defined to have the following meanings:

The terminology used herein is for the purpose of describing particular embodiments only and is not intended to be limiting. As used herein, the singular forms “a,” “an” and “the” are intended to include the plural forms as well, unless the context clearly indicates otherwise. Thus, for example, reference to “a natural killer cell” includes a plurality of natural killer cells.

All numerical designations, e.g., pH, temperature, time, concentration, amounts, and molecular weight, including ranges, are approximations which are varied (+) or (−) by increments of 0.1 or 1.0, where appropriate. It is to be understood, although not always explicitly stated, that all numerical designations may be preceded by the term “about.”

The term “about” when used in conjunction with a value means any value that is reasonably close to the value, i.e., within the range of +10% of the value. In particular, it would include the value itself.

Unless otherwise noted, a percentage, when denoting a concentration, refer to a w/w percentage.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

It is also to be understood, although not always explicitly stated, that the reagents described herein are merely exemplary and that equivalents of such are known in the art.

“Optional” or “optionally” means that the subsequently described event or circumstance can or cannot occur, and that the description includes instances where the event or circumstance occurs and instances where it does not.

The term “comprising” is intended to mean that the compositions and methods include the recited elements, but not excluding others. “Consisting essentially of,” when used to define compositions and methods, shall mean excluding other elements of any essential significance to the combination. For example, a composition consisting essentially of the elements as defined herein would not exclude other elements that do not materially affect the basic and novel characteristic(s) of the claims. “Consisting of” shall mean excluding more than trace amount of other ingredients and substantial method steps. Embodiments defined by each of these transition terms are within the scope of the disclosure.

As used herein, “natural killer (NK) cells” are cells of the immune system that kill target cells in the absence of a specific antigenic stimulus, and without restriction according to major histocompatibility complex (MHC) class. Target cells may be cancer or tumor cells. NK cells are characterized by the presence of CD56 and the absence of CD3 surface markers.

As used herein, “NK-92® cells” refer to natural killer cells derived from the highly potent unique cell line described in Gong et al. (1994), rights to which are owned by NantKwest. As used herein, the term “NK-92,” “NK-92 cells,” “NK-92®” or “NK-92® cells” is intended to refer to the original NK-92® cell lines as well as clones of NK-92® cells, and NK-92® cells that have been modified (e.g., by introduction of exogenous genes). NK-92® cells and exemplary and non-limiting modifications thereof are described in U.S. Pat. Nos. 7,618,817; 8,034,332; 8,313,943; 9,181,322; 9,150,636; and published U.S. application Ser. No. 10/008,955, all of which are incorporated herein by reference in their entireties, and include wild type NK-92®, NK-92®-CD16, NK-92®-CD16-Y, NK-92®-CD16-ζ, NK-92®-CD16 (F176V), NK-92®MI, and NK-92®CI. NK-92® cells are known to persons of ordinary skill in the art, to whom such cells are readily available from NantKwest, Inc.

As used herein, the term “aNK™ cells” or “aNK cells” refers to unmodified natural killer cells (i.e., parental NK-92® cells) derived from the highly potent unique cell line described in Gong et al. (1994), rights to which are owned by NantKwest.

As used herein, the term “haNK® cells” or “haNK cells” refers to NK-92® cells that were modified to express CD16 (or Fc receptor) on the cell surface (hereafter, “CD16+NK-92® cells” or “haNK® cells”).

As used herein, the term “taNK cells,” “taNK® cells,” or “CAR-modified NK-92® cells” refers to NK-92® cells that have been engineered to express a chimeric antigen receptor (CAR) with affinity for a cancer specific antigen, a cancer associated antigen, or a tumor specific antigen. In some embodiments, the tumor specific antigen is HER-2, e.g., human HER-2, and these NK-92® cells are referred to as HER-2 taNK cells.

As used herein, the term “t-haNK cells” or “t-haNK™ cells” refers to NK-92® cells that have been engineered to express (1) a chimeric antigen receptor (CAR) with affinity for a cancer specific antigen, a cancer associated antigen, or a tumor specific antigen; and (2) a Fc receptor. In some embodiments, the tumor specific antigen is CD19, e.g., human CD19, and these NK-92® cells are referred to as CD19 t-haNK cells. In some embodiments, the tumor specific antigen is PD-L1. In some embodiments, the t-haNK cells express a chimeric antigen receptor PD-L1 CAR that has a sequence of SEQ ID NO: 5. In some embodiments, the t-haNK cells express a chimeric antigen receptor CD19 CAR that has a sequence of SEQ ID NO: 6. In some embodiments, the t-haNK cells express a chimeric antigen receptor HER2 CAR that has a sequence of SEQ ID NO: 7.

The term “Fc receptor” refers to a protein found on the surface of certain cells (e.g., natural killer cells) that contribute to the protective functions of the immune cells by binding to part of an antibody known as the Fc region. Binding of the Fc region of an antibody to the Fc receptor (FcR) of a cell stimulates phagocytic or cytotoxic activity of a cell via antibody-mediated phagocytosis or antibody-dependent cell-mediated cytotoxicity (ADCC). FcRs are classified based on the type of antibody they recognize. For example, Fc-gamma receptors (FcγR) bind to the IgG class of antibodies. FcγRIII-A (also called CD16) is a low affinity Fc receptor bind to IgG antibodies and activate ADCC. FcγRIII-A are typically found on NK cells. NK-92® cells do not express FcγRIII-A.

The term “chimeric antigen receptor” (CAR), as used herein, refers to an extracellular antigen-binding domain that is fused to an intracellular signaling domain. CARs can be expressed in T cells or NK cells to increase cytotoxicity. In general, the extracellular antigen-binding domain is a scFv that is specific for an antigen found on a cell of interest. A CAR-expressing NK-92® cell is targeted to cells expressing certain antigens on the cell surface, based on the specificity of the scFv domain. The scFv domain can be engineered to recognize any antigen, including tumor-specific antigens.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, a polynucleotide encompasses both the double-stranded form and each of two complementary single-stranded forms known or predicted to make up the double-stranded form.

The term “expression” refers to the production of a gene product. The term “transient” when referred to expression means a polynucleotide is not incorporated into the genome of the cell.

The term “cytokine” or “cytokines” refers to the general class of biological molecules which effect cells of the immune system. Exemplary cytokines include, but are not limited to, interferons and interleukins (IL), in particular IL-2, IL-12, IL-15, IL-18 and IL-21. In preferred embodiments, the cytokine is IL-2.

As used herein, the term “vector” refers to a non-chromosomal nucleic acid comprising an intact replicon such that the vector may be replicated when placed within a permissive cell, for example by a process of transformation. A vector may replicate in one cell type, such as bacteria, but have limited ability to replicate in another cell, such as mammalian cells. Vectors may be viral or non-viral. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

As used herein, the term “substantially the same”, used interchangeably with the term “comparable”, or “similar”, when referring to cytotoxicity, viability, cell recovery, or cell doubling time refers to the that the two measurements of cytotoxicity, viability, cell recovery, or cell doubling time are no more than 25%, no more than 20%, no more than 15% different, no more than 10%, no more than 8%, or no more than 5% different from each other.

As used herein, the terms “cytotoxic” when used to describe the activity of effector cells such as NK cells, relates to killing of target cells by any of a variety of biological, biochemical, or biophysical mechanisms.

As used herein, the term “harvesting” refers to separating and collecting cells from their culture medium and preparing the cells for therapeutic applications. Harvesting comprises washing cells with a suitable buffer, e.g., 5% albumin, and optionally resuspending cells in a buffer that is suitable for intended applications, e.g., for infusion.

As used herein, the term “recovery” refers to relative amount of the cells obtained after the harvesting process is completed as compared to the number of cells entering the harvesting process. In some cases, recovery is expressed as a percentage, for example, when continuous centrifugation is used as a means for harvesting cells, recovery can be expressed as the following equation:

Recovery=the amount of cells recovered from continuous centrifugation/amount of cells entered

into continuous centrifugation.

Titles or subtitles may be used in the specification for the convenience of a reader, which are not intended to influence the scope of the present disclosure. Additionally, some terms used in this specification are more specifically defined below.

### Methods for Preparing NK-92® Cells

### Methods for Storing NK-92® Cells

Provided herein are methods for preparing NK-92® cells for storage, for example, by cryopreservation. This disclosure provides a cell freezing medium for cryopreserving NK-92® cells, which comprises a first composition and a second composition. As compared to various existing freezing media, the cell freezing medium of this disclosure can mitigate temperature-induced molecular cell stress responses during freezing and thawing. It can be used to cryopreserve a broad spectrum of cell types, including NK-92® cells. The medium is much more effective in reducing post-preservation necrosis and apoptosis as compared to commercial and home-brew isotonic and extracellular formulations. This enables greatly improved post-thaw cell yield, viability, and recovery. Optionally, one or more or all ingredients in the first and/or second compositions meet the USP standard. Optionally, these [0061] The first composition disclosed herein comprises one or more electrolytes. Exemplary electrolytes that can be used in the first composition may be one or more of potassium ions, sodium ions, magnesium ions, and calcium ions. Optionally, potassium ions are present in the first composition in a concentration ranging from 35-45 mM, sodium ions are present in a concentration ranging from 80-120 mM, magnesium ions are present in a concentration ranging from 2-10 mM, and calcium ions present in a concentration ranging from 0.01-0.1 mM.

The first composition disclosed herein also comprises one or more macromolecular agents. Exemplary macromolecular agents that can be used in the first composition may be one or more of polysaccharide and colloidal starch. Optionally, the first composition comprises human albumin.

The first composition may comprise a pH buffer that is effective under physiological and hypothermic conditions. Non-limiting examples of suitable pH buffers include dihydrogen phosphate (H2PO₄-), bicarbonate ion (HCO3-) and HEPES. Optionally, H2PO₄- is present in an amount of 5-15 mmol/L, e.g., 10.0 mmol/L, HCO3- is present in an amount of 2.5-10 mmol/L, e.g., 5.0 mmol/L, and/or HEPES is present in an amount of 15-35 mmol/L.

The first composition may also comprise one or more sugars. Optionally, the one or more sugars comprises sucrose. Optionally, the one or more sugar is a simple sugar that is selected from the group consisting of glucose, dextran, dextrose, trehalose, and maltose. Optionally, the glucose is present in the first composition at a concentration of 2.0-10.0 mmol/L, e.g., 3.0-7.0 mmol/L, or 5.0 mmol/L.

The first composition may also comprise one or more sugar alcohols. Each of the sugar alcohols can be a 3-carbon, 4-caron, 5-carbon, 6-carbon, 7-carbon, 12-carbon, 18-carbon, or 24-carbon sugar alcohol. Optionally, the sugar alcohol is a 6-carbon sugar alcohol. Non-limiting examples of 6-carbon sugar alcohols include mannitol, sorbitol, galactitol, fucitol, iditol, and inositol. Optionally, the 6-carbon sugar alcohol is mannitol. Non-limiting examples of 3-carbon sugar alcohols include glycerol, erythritol, and threitol. Non-limiting examples of 5-carbon sugar alcohols include arabitol, xylitol, ribitol. Non-limiting examples of 7-carbon sugar alcohols include volemitol. Non-limiting examples of 12-carbon sugar alcohols include isomalt, maltitol, and lactitol. Non-limiting examples of 18-carbon sugar alcohols include maltotriitol. Non-limiting examples of 24-carbon sugar alcohols include maltotetraitol. Optionally, the one or more sugar alcohols are present in the the first composition in a total amount of 10-30 mmol/L, e.g., 20.0 mmol/L.

The first composition may also comprise one or more impermeant agents to maintain ionic and osmotic balance within body tissues during hypothermia. These impermeant agents is include to balance the fixed ions inside cells that are responsible for ocotic pressure leading to osmotic cell swelling and eventual lysis during hypothermia. Non-limiting examples of impermeant agents include lactobionate, gluconate, citrate and glycerophosphate. Lactobionate, for example, is a strong chelator of calcium and iron and may contribute to minimizing cell injury due to calcium influx and free radical formation. Optionally, the one or more impermeant agents are present in a total concentration of 40-200 mmol/L, e.g., 50-150 mmol/L, e.g., 100.0 mmol/L.

The first composition also comprises DMSO. Optionally, the DMSO is USP grade DMSO. The USP is a chemical grade of sufficient purity to meet or exceed requirements of the United States Pharmacopeia (USP) and is acceptable for food, drug, or medical use. Optionally, the first composition comprises 2-18% v/v DMSO, e.g., 4-14%, 6-12%, 6-16%, or about 10% v/v DMSO.

Optionally, the first composition comprises a substrate effective for regeneration of ATP. For example, the substrate may be one that is selected from the group consisting of adenosine, fructose, ribose and adenine. Optionally, the first composition comprises adenosine. Optionally adenosine is present in a concentration of 0.5-5 mmol/L, e.g., 1-4 mmol/L, 1-3 mmol/L, or about 2.0 mmol/L.

Optionally, the first composition comprises a cellular antioxidant, such as glutathione. In addition to acting as an important cellular antioxidant, glutathione is also a cofactor for glutathione peroxidase, which enables metabolism of lipid peroxides and hydrogen peroxide. Optionally, the first composition comprises 0.5-5 mmol/L of the cellular antioxidant, e.g., 1-4 mmol/L, 2-5 mmol/L, or about 3.0 mmol/L. For the purpose of this disclosure, the substrate and the antioxidant used in the medium are collectively referred to as the metabolites.

Suitable medium for use as the first composition include Cryostor® CS10, available from Biolife Solutions, Bothell, WA.

In one illustrative embodiment, the first composition comprises the following ingredients as shown in Table 1 below.

TABLE 1

| | Components | Concentrations | |
|---|---|---|---|
| Electrolytes | Na⁺ | 100.0 | mmol/L |
| | K⁺ | 42.5 | mmol/L |
| | Ca²⁺ | 0.05 | mmol/L |
| | Mg²⁺ | 5.0 | mmol/L |
| | Cl⁻ | 17.1 | mmol/L |
| pH buffers | H₂PO₄⁻ | 10.0 | mmol/L |
| | HCO₃⁻ | 5.0 | mmol/L |
| | HEPES | 25.0 | mmol/L |
| Impermeants | Lactobionate | 100.0 | mmol/L |
| Sugar alcohol | Mannitol | 20.0 | mmol/L |
| Sugars | Glucose | 5.0 | mmol/L |
| | Dextran-40 | 6.0% | |
| | Sucrose | 20.0 | mmol/L |
| Metabolites | Adenosine | 2.0 | mmol/L |
| | Glutathione | 3.0 | mmol/L |

| | | |
|---|---|---|
| DMSO | DMSO | 10% |

In this particular embodiment, the first composition has an osmolarity of 350 mOsm/kg and a pH of 7.6 (at 25° C.).

The second composition used in the cell freezing medium include albumin. Albumin is a protein supplement in cell culture used to deliver unesterified fatty acids into and from cells; albumin can be, for example, human albumin, such as human serum albumin (HSA) and human plasma albumin. Albumin is beneficial to the overall health of the cryopreserved cells. Optionally, the albumin is human albumin. Optionally, the albumin is human serum albumin or plasma derived human albumin. Human albumin (“HA”) is the most copious protein in human serum at approximately 3.5-5.0 g/dL and functions as a carrier protein for steroids and fatty acids in blood. Human albumin is commercially available, for example, from CSL Behring. Optionally, the second composition comprises 1-10% w/v HA, e.g., 2-10%, 2-8%, 3-6%, or 5.0% w/v. As discussed below, including HA in the cell freezing medium not only is beneficial for maintaining cell morphology and function, it can also ease manufacturing process.

The cell freezing medium disclosed herein is made by mixing the first composition and the second composition. During the mixing, the DMSO in the first composition is diluted. Thus, the cell freezing medium typically comprises a low DMSO concentration, e.g., less than 10%. In general, DMSO, although widely used in cell freezing medium, is cytotoxic; it can alter cell morphology, reduce membrane integrity, and reduce cell viability, the toxic effect worsens as the concentration of DMSO increases in the medium. Thus, the claimed methods can maintain the DMSO at a relatively low concentration, e.g., at a concentration that is lower than 10%, which can improve post-thaw cell viability and function. Optionally, the first composition and the second composition in the cell freezing medium are mixed in a volume ratio of 1:5 to 5:1, e.g., 1:3 to 3:1, 1:2 to 2:1, or about 1:1. Optionally, the cell freezing medium comprises about 5, about 6, about 7, about 8, about 9, or about 10% v/v DMSO.

Thus, as further disclosed below, the NK-92® cells that have been freezed in cryopreservation medium comprising the first composition and the second composition disclosed herein shows improved viability, better cytotoxicity, and/or ADCC than cells preserved in other media and are suitable for direct infusion to a patient in need.

This disclosure provides methods of freezing NK-92® cells using the novel cell freezing medium disclosed herein. In some cases, NK-92® cells can be harvested from culture by centrifugation. In some cases, the cell culture before centrifugation contains 2-10%, e.g., about 5% HA. In some cases, the cell culture before harvesting contains about the same concentration of the HA as in the second composition. Optionally the centrifugation is performed in a kSep®400 centrifuge that are aseptically attached to a culture vessel, e.g., Xuri bags. In some cases, harvested cells are resuspended in the second composition before adding the first composition. As the cell culture before harvesting typically already contains HA, using the second composition, which also comprises HA, keeps cells in a similar medium environment and thus minimizes negative impact of harvesting on the morphology and function of the cells. The methods dispense the need for washing cells after harvesting, an extra step often required when before freezing cells in a conventional cell freezing medium (which typically has different medium composition from the pre-harvesting medium). Thus, the claimed method significantly reduces processing time, reduces operational errors and increases cell recovery.

Optionally, the cells in the cell freezing medium are in a concentration that is suitable for clinical applications. For example, the NK-92® cells may be in a concentration of 1.8-2.4×10e7 cells/mL, e.g., 1.8×10e7 cells/mL, or 2×10e7 cells/mL. Optionally, the NK-92® cells are in the cell freezing medium in a volume that is suitable for infusion to patients. For example, the volume may be 55-100 mL

NK-92® cells in the cell freezing medium may be frozen using methods well known in the art. In one illustrative example, the NK-92® cells are frozen using a controlled-rate freezing program, until the temperature reaches −70° C. to −196° C., e.g., −80° C. to −196° C. Frozen cells are then stored in vapor phase of a liquid N2 cryofreezer.

Cells can be thawed by any methods that are suitable for thawing cells. Optionally, the cells can be thawed at 37° C., in a device such as a waterbath or ViaThaw control rate thawing unit from Asymptote, Cambridge, United Kingdom.

### Methods of Harvesting NK-92® Cells

Growing NK-92® cells typically starts from thawing frozen NK-92® cells and seeding them in a container with a suitable medium. Cells are allowed to recover until the cell viability reaches a certain value, for example, greater than 85%. Cells are then expanded in a vessel, e.g., a G-Rex flask, to a desirable cell density, for example, a density that is equal to or less than 1.2×106 cells/mL. The cell culture from the vessel is then collected and used to inoculate one or more larger culture vessels. Commonly used such larger culture vessels include Xuri bags, which can have a volume of at least 2 liters, at least 10 liters, or at least 50 liters. Transfer of cells between the different vessels can be performed using means well known in the art, e.g., using a pump or a gravity feed, performed under sterile conditions.

NK-92® cells so produced can be harvested by centrifugation. Optionally the centrifugation is performed in a continuous centrifuge that are aseptically attached to the culture vessel, e.g., the Xuri bags, that is at the end of the expansion process. Continuous centrifugation refers to a centrifugation of a duration of 45-60 min, depending on the cell culture volume, to concentrate the cells, followed by a cell wash of at least 1 min, at least 3 min, or at least 5 min. The culture supernatant is then removed and the cells are resuspended in a wash buffer. Optionally, the wash can be repeated for at least two times, at least three times, e.g., 4-6 times. After the final wash, the mixture containing the cells and wash buffer can be centrifuged again and the cells are collected and processed for therapeutic applications.

In some embodiments, the wash buffer comprises albumin. In some embodiments, the wash buffer comprises 1-5% albumin, e.g., 2-5%, 3-5%, or 4-5%, preferably 5% albumin. Albumin is a protein supplement in cell culture used to deliver unesterified fatty acids into and from cells; Albumin can be derived from human or non-human sources, for example, human or bovine. Human albumin can be derived from human serum (“human serum albumin”) or human plasma (“human plasma albumin”), or can be synthesized in vitro, e.g., by expressing a gene (e.g., sequence of NM_000477) encoding the human albumin. To date, albumin, especially albumin derived from human has not been used for washing cells during harvesting because it is relatively costly as compared to other wash buffers, such as PBS or growth media, such as X VIVO™ 10. Human Albumin is commercially available, for example, from CSL Behring.

In addition to albumin, the wash buffer may also comprise 1-10 mg/mL sodium, e.g., 3-5 mg/mL sodium, or 3.2 mg/mL sodium. Optionally, the wash buffer lacks sugar, e.g., dextran. Optionally, the wash buffer lacks dextran-40.

The method of harvesting can recover 80 to 100% of NK-92® cells, e.g., 85-99%, or 89-99% of NK-92® cells. The yield of harvesting can be assessed using standard cell counting procedure, e.g., a trypan blue dye-exclusion method or a Nucleocounter NC-200 method. Optionally, the method of harvesting using the methods disclosed herein can recover substantially the same amount of NK-92® cells as the method of harvesting using X-VIVO™10 medium.

### Methods for Evaluating NK-92® Cells

Optionally, the viability of the thawed cells is assessed. Methods for measuring cell viability are also well known, for example, trypan blue exclusion assay, in which dead cells are stained blue and viable cell number can be calculated by substracting the trypan blue stained cells from the total cells. Cell counting can be performed on a counting chamber of a hemacytometer. Automatic cell counting, based on the fact that cells show great electrical resistance, are also commonly used to count cells as well as measure their volume. One example of the automatic cell counter is the Coulter counter, available from Beckman Coulter. Another example of the automatic cell counter is NucleoCounter® NC-200™, available from Chemometec. The NK-92® cells that have been freezed in the cell freezing medium described herein and thawed may have 80-100%, e.g., 85-100%, 90-100%, 92-100% viability. Optionally, the NK-92® cells that have been freezed and thawed may have viability that is at least 90%, at least 91%, at least 92%, at least 93%, at least 94% of that of the cells before freezing.

Optionally, the cytotoxicity of the thawed NK-92® cells can be assessed. Direct cytotoxicity of the produced NK-92® cells, the ability to target and kill aberrant cells, such as virally infected and tumorigenic cells, can be assessed by methods well known in the art, for example, a 51Cr release assay (Gong et al. (1994)) using the procedure described by Klingemann et al. (Cancer Immunol. Immunother. 33:395-397 (1991)). The percentage of specific cytotoxicity can be calculated based on the amount of released 51Cr. See Patent Pub. No. US20020068044.

Alternatively, direct cytotoxicity of the produced NK-92® cells can also be assessed using a calcein release assay. For example, the NK-92® cells (referred to as the effector in the assay) can be mixed with the calcein loaded target cells (referred to as target in the assay) at certain ratios. After incubation for a period of time (e.g., 2-8 hours, or about 3 hours) the calcein released from the target cells can be assessed, e.g., by a fluorescence plate reader. The ratio of the effector and target used in the assay may vary, optionally the effector: target ratio may be 20:1, 15:1, 10:1, 8:1, or 5:1; preferably the effector: target ratio is 10:1. The target cells can be any cells that express MHC molecules that can be recognized by the NK-92® cells, for example, K562 cells, or BT-474 cells. The values of cytotoxicity of NK-92® cells may vary depending on the type of target cells used as well as the effector: target ratio and the growth cycle which the NK-92® cells are in.

The NK-92® cells after thawing has surprisingly retained the substantial portion of the cytotoxicity; in some cases, the thawed NK-92® cells at least 70-100%, e.g., 80-100%, at 90-100% of the cytotoxicity of that of the NK-92® cells before the cells are frozen (pre-freeze state). Optionally, the thawed NK-92® cells have 70-100%, e.g., 75-95%, 70-90%, or about 88%, direct cytotoxicity, when using a calcein release assay as described below. Optionally, the effect: target ratio is 10:1 and the target cells being K562 cells.

Optionally, the cytotoxicity of NK-92® cells, e.g., haNK& cells, that is assessed is the antibody dependent cytotoxicity (ADCC). Methods for measuring the ADCC of NK-92® cells are similar to the methods of measuring direct cytotoxicity as described above except that an antibody that can recognize the target cell is added. The Fc receptor of the NK cells recognizes the cell-bound antibodies and triggers cytolytic reaction and killing the target cells. In one illustrative example, the haNK® cells can be incubated with Rituxan (an antibody) and Ramos (target cells) and killing of the Ramos cells can be measured by the release of internal components of the target cells, e.g., 51Cr or calcein, as described above. The ratio of the effector and target used in the assay may vary, optionally the effector: target ratio may be 20:1, 15:1, 10:1, 8:1, or 5:1; preferably the effector: target ratio is 10:1. in some cases, the thawed haNK® cells have 70-100%, e.g., 80-100%, at 90-100% of the cytotoxicity of that of the NK-92® cells before the cells are frozen (pre-freeze state). HaNK® cells after thawing may have a ADCC cytotoxicity of 80-120%, e.g., 90-115%, 97-110%, or 100-120% when using an effector: target ratio of 10:1. In some embodiments, the haNK® cells after thawing may have a ADCC cytotoxicity that is 80-100% of that of the haNK® cells before the cells are frozen.

Optionally, the phenotypes of the thawed cells are assessed by assaying the expression level of one or more surface markers, optionally from of CD54, CD56, NKG2D, NKp30, CD3, and CD16. The marker expression can be determined using methods well known in the art, e.g., by staining cells with specific fluorochrome-conjugated antibodies and detecting bound antibodies by flow cytometry. The freeze and thaw of NK-92® cells in the cell freezing medium disclosed herein does not alter the expression of key surface makers. For example, the NK-92® cells freezed and thawed as described above retained expression of surface markers typical of an NK cell in an early differentiation stage, such as a number of activation receptors including NKG2D and NKp30, but lacking FcγRIIIa (CD16). The thawed NK-92® cells also express CD56 and CD54. Optionally, greater than 90% of the cells in the population of the thawed cells express CD56, CD54, NKG2D, or NKp30 and less than 5% of the cells in the population of cells express CD3 or CD16.

NK-92® cells harvested using 1-5% albumin as disclosed herein may have substantially the same cytotoxicity as control NK-92® cells that have been grown in the same condition but have not been harvested. The control cells can be, for example, the NK-92® cells from the G-Rex flask. The NK-92® cells harvested using the method disclosed herein can also have substantially the same cytotoxicity as NK-92® cells that have been harvested in X-VIVO™10 medium. The NK-92® cells that have been harvested using the methods disclosed herein may have substantially the same cytotoxicity as the NK-92® cells before harvesting.

Cytotoxicity of NK-92® cells can be reflected by their direct cytotoxicity or ADCC. Direct cytotoxicity of the produced NK-92® cells, the ability to target and kill aberrant cells, such as virally infected and tumorigenic cells, can be assessed by methods well known in the art, for example, a 51Cr release assay, (Gong et al. (1994)) using the procedure described by Klingemann et al. (Cancer Immunol. Immunother. 33:395-397 (1991)). Briefly, 51Cr-labeled target cells are mixed with NK-92® cells and are lysed. The percentage of specific cytotoxicity can be calculated based on the amount of released 51Cr. See Patent Pub. No. US20020068044.

Alternatively, direct cytotoxicity of the produced NK-92® cells can also be assessed using a calcein release assay. For example, the NK-92® cells (referred to as the effector in the assay) can be mixed with the calcein loaded target cells (referred to as target in the assay) at certain ratios. After incubation for a period of time, the calcein released from the target cells can be assessed, e.g., by a fluorescence plate reader. The ratio of the effector and target used in the assay may vary, optionally the effector: target ratio may be 20:1, 15:1, 10:1, 8:1, or 5:1; preferably the effector: target ratio is 10:1. The target cells can be any cells that express MHC molecules that can be recognized by the NK-92® cells, for example, K562 cells, or BT-474 cells. The values of cytotoxicity of NK-92® cells may vary depending on the type of target cells used as well as the effector: target ratio. In general, the NK-92® cells produced using the methods described herein can have a cytotoxicity of 60-100%, e.g., 70-100% or 80-100%. In some cases, aNK cells may have a cytotoxicity of 80-100% when using K562 cells as the target cells, e.g., 82-100%, 85-100%, 87-100%, 88-100%, or 89-100%, by a calcein release assay.

Optionally, the cytotoxicity of NK-92® cells, e.g., haNK cells, that is assessed is the antibody dependent cytotoxicity (ADCC). Methods for measuring the ADCC of NK-92® cells are similar to the methods of measuring direct cytotoxicity as described above except that an antibody that can recognize the target cell is added. The Fc receptor of the NK cells recognizes the cell-bound antibodies and triggers cytolytic reaction and killing the target cells. In one illustrative example, the haNK cells can be incubated with Rituxan (an antibody) and Ramos (target cells) and killing of the Ramos cells can be measured by the release of internal components of the target cells, e.g., 51Cr or calcein, as described above.

### NK-92® Cells

The NK-92® cells that can be cultured using the methods disclosed herein include aNK cells, haNK cells, taNK and t-haNK cells, which are further described below.

The NK-92® cell line is a unique cell line that was discovered to proliferate in the presence of interleukin 2 (IL-2). Gong et al., Leukemia 8:652-658 (1994). These cells have high cytolytic activity against a variety of cancers. The NK-92® cell line is a homogeneous cancerous NK cell population having broad anti-tumor cytotoxicity with predictable yield after expansion. Phase I clinical trials have confirmed its safety profile. NK-92® was discovered in the blood of a subject suffering from a non-Hodgkins lymphoma and then immortalized ex vivo. NK-92® cells are derived from NK cells, but lack the major inhibitory receptors that are displayed by normal NK cells, while retaining the majority of the activating receptors. NK-92® cells do not, however, attack normal cells nor do they elicit an unacceptable immune rejection response in humans. Characterization of the NK-92® cell line is disclosed in WO 1998/49268 and U.S. Patent Application Publication No. 2002-0068044.

The NK-92® cell line is found to exhibit the CD56^{bright}, CD2, CD7, CD11a, CD28, CD45, and CD54 surface markers. It furthermore does not display the CD1, CD3, CD4, CD5, CD8, CD10, CD14, CD16, CD19, CD20, CD23, and CD34 markers. Growth of NK-92® cells in culture is dependent upon the presence of recombinant interleukin 2 (rIL-2), with a dose as low as 1 IU/mL being sufficient to maintain proliferation. IL-7 and IL-12 do not support long-term growth, nor do other cytokines tested, including IL-1α, IL-6, tumor necrosis factor α, interferon α, and interferon γ. NK-92® has high cytotoxicity even at a low effector: target (E:T) ratio of 1:1. Gong, et al., supra. NK-92® cells are deposited with the American Type Culture Collection (ATCC), designation CRL-2407.

Heretofore, studies on endogenous NK cells have indicated that IL-2 (1000 IU/mL) is critical for NK cell activation during shipment, but that the cells need not be maintained at 37° C. and 5% carbon dioxide. Koepsell, et al., Transfusion 53:398-403 (2013).

Modified NK-92® cells are known and include, but are not limited to, those described in, e.g., U.S. Pat. Nos. 7,618,817, 8,034,332, and 8,313,943, US Patent Application Publication No. 2013/0040386, all of which are incorporated herein by reference in their entireties, such as wild type NK-92®, NK-92®-CD16, NK-92®-CD16-γ, NK-92®-CD16-ζ, NK-92®-CD16 (F157V), NK-92®mi and NK-92®ci.

Although NK-92® cells retain almost all of the activating receptors and cytolytic pathways associated with NK cells, they do not express CD16 on their cell surfaces. CD16 is an Fc receptor which recognizes and binds to the Fc portion of an antibody to activate NK cells for antibody-dependent cellular cytotoxicity (ADCC). Due to the absence of CD16 receptors, NK-92® cells are unable to lyse target cells via the ADCC mechanism and, as such, cannot potentiate the anti-tumor effects of endogenous or exogenous antibodies (i.e., Rituximab and Herceptin).

Studies on endogenous NK cells have indicated that IL-2 (1000 IU/mL) is critical for NK cell activation during shipment, but that the cells need not be maintained at 37° C. and 5% carbon dioxide. Koepsell, et al., Transfusion 53:398-403 (2013). However, endogenous NK cells are significantly different from NK-92® cells, in large part because of their distinct origins: NK-92® is a cancer-derived cell line, whereas endogenous NK cells are harvested from a donor (or the patient) and processed for infusion into a patient. Endogenous NK cell preparations are heterogeneous cell populations, whereas NK-92® cells are a homogeneous, clonal cell line. NK-92® cells readily proliferate in culture while maintaining cytotoxicity, whereas endogenous NK cells do not. In addition, an endogenous heterogeneous population of NK cells does not aggregate at high density. Furthermore, endogenous NK cells express Fc receptors, including CD-16 receptors that are not expressed by NK-92® cells.

### Fc Receptors

In some embodiments NK-92® cells are modified to express an Fc receptor protein on the cell surface. Fc receptors bind to the Fc portion of antibodies. Several Fc receptors are known (for example, as provided in Table 2 below), and differ according to their preferred ligand, affinity, expression, and effect following binding to the antibody.

TABLE 2
Illustrative Fc receptors

| | Principal | Affinity | | |
|---|---|---|---|---|
| Receptor | antibody | for | | Effect following binding |
| name | ligand | ligand | Cell distribution | to antibody |
| FcγRI (CD64) | IgG1 and | High | Macrophages | Phagocytosis |
| | IgG3 | (Kd~10⁻⁹M) | Neutrophils | Cell activation |
| | | | Eosinophils | Activation of respiratory |
| | | | Dendritic cells | burst |
| | | | | Induction of microbe |
| | | | | killing |
| FcγRIIA (CD32) | IgG | Low | Macrophages | Phagocytosis |
| | | (Kd > 10⁻⁷M) | Neutrophils | Degranulation (eosinophils) |
| | | | Eosinophils | |
| | | | Platelets | |
| | | | Langerhans cells | |
| FcγRIIB1 (CD32) | IgG | Low | B Cells | No phagocytosis |
| | | (Kd > 10⁻⁷M) | Mast cells | Inhibition of cell activity |
| FcγRIIB2 (CD32) | IgG | Low | Macrophages | Phagocytosis |
| | | (Kd > 10⁻⁷M) | Neutrophils | Inhibition of cell activity |
| | | | Eosinophils | |
| FcγRIIIA (CD16a) | IgG | Low | NK cells | Induction of antibody- |
| | | (Kd > 10⁻⁶M) | Macrophages (certain | dependent cell-mediated |
| | | | tissues) | cytotoxicity (ADCC) |
| | | | | Induction of cytokine |
| | | | | release by macrophages |
| FcγRIIIB (CD16b) | IgG | Low | Eosinophils | Induction of microbe |
| | | (Kd > 10⁻⁶M) | Macrophages | killing |
| | | | Neutrophils | |
| | | | Mast cells | |
| | | | Follicular dendritic | |
| | | | cells | |
| FcεRI | IgE | High | Mast cells | Degranulation |
| | | (Kd ~10⁻¹⁰M) | Eosinophils | Phagocytosis |
| | | | Basophils | |
| | | | Langerhans cells | |
| | | | Monocytes | |
| FcεRII (CD23) | IgE | Low | B cells | Possible adhesion molecule |
| | | (Kd > 10⁻⁷M) | Eosinophils | IgE transport across human |
| | | | Langerhans cells | intestinal epithelium |
| | | | | Positive-feedback |
| | | | | mechanism to enhance |
| | | | | allergic sensitization (B |
| | | | | cells) |
| FcαRI (CD89) | IgA | Low | Monocytes | Phagocytosis |
| | | (Kd > 10⁻⁶M) | Macrophages | Induction of microbe |
| | | | Neutrophils | killing |
| | | | Eosinophils | |
| Fcα/μR | IgA and IgM | High for | B cells | Endocytosis |
| | | IgM, | Mesangial cells | Induction of microbe |
| | | Mid for | Macrophages | killing |
| | | IgA | | |
| FcRn | IgG | | Monocytes | Transfers IgG from a |
| | | | Macrophages | mother to fetus through the |
| | | | Dendritic cells | placenta |
| | | | Epithelial cells | Transfers IgG from a |
| | | | Endothelial cells | mother to infant in milk |
| | | | Hepatocytes | Protects IgG from |
| | | | | degradation |

In some embodiments NK-92® cells are modified to express an Fc receptor protein on the cell surface.

In some embodiments, the Fc receptor is CD16. A representative amino acid sequence encoding CD16 is shown in SEQ ID NO:2. A representative polynucleotide sequence encoding CD16 is shown in SEQ ID NO:1. In some embodiments, NK-92® cells are modified by introducing a polynucleotide encoding a CD16 polypeptide has at least about 70% polynucleotide sequence identity with a polynucleotide sequence encoding a full-length, including signal peptide, naturally occurring CD16 that has a phenylalanine at position 176 of the full-length CD16. In some embodiments, a polynucleotide encoding a CD16 polypeptide has at least about 70% polynucleotide sequence identity with a polynucleotide sequence encoding a full-length, including the signal peptide, naturally occurring CD16 that has a valine at position 176.

Homologous polynucleotide sequences include those that encode polypeptide sequences coding for variants of CD16. In some embodiments, homologous CD16 polynucleotides may be about 150 to about 700, about 750, or about 800 polynucleotides in length, although CD16 variants having more than 700 to 800 polynucleotides are within the scope of the disclosure.

In other examples, cDNA sequences having polymorphisms that change the CD16 amino acid sequences are used to modify the NK-92® cells, such as, for example, the allelic variations among individuals that exhibit genetic polymorphisms in CD16 genes. In other examples, CD16 genes from other species that have a polynucleotide sequence that

In examples, variant polypeptides are made using methods known in the art such as oligonucleotide-mediated (site-directed) mutagenesis, alanine scanning, and PCR mutagenesis. Site direct mutagenesis (Carter, 1986; Zoller and Smith, 1987), cassette mutagenesis, restriction selection mutagenesis (Wells et al., 1985) or other known techniques can be performed on the cloned DNA to produce CD16 variants (Ausubel, 2002; Sambrook and Russell, 2001).

Conservative substitutions in the amino acid sequence of human CD16 polypeptide, whereby an amino acid of one class is replaced with another amino acid of the same class, fall within the scope of the disclosed CD16 variants as long as the substitution does not materially alter the activity of the polypeptide. Conservative substitutions are well known to one of skill in the art. Non-conservative substitutions that affect (1) the structure of the polypeptide backbone, such as a β-sheet or α-helical conformation, (2) the charge, (3) the hydrophobicity, or (4) the bulk of the side chain of the target site can modify CD16 polypeptide function or immunological identity. Non-conservative substitutions entail exchanging a member of one of these classes for another class. Substitutions may be introduced into conservative substitution sites or more preferably into non-conserved sites.

In some embodiments, CD16 polypeptide variants are at least 200 amino acids in length and have at least 70% amino acid sequence identity, or at least 80%, or at least 90% identity to SEQ ID NO:1 or SEQ ID NO:2. In some embodiments, CD16 polypeptide variants are at least 225 amino acid in length and have at least 70% amino acid sequence identity, or at least 80%, or at least 90% identity to SEQ ID NO:1 or SEQ ID NO:2.

In some embodiments a nucleic acid encoding a CD16 polypeptide may encode a CD16 fusion protein. A CD16 fusion polypeptide includes any portion of CD16 or an entire CD16 fused with a non-CD16 polypeptide. In some embodiment, a fusion polypeptide may be created in which a heterologous polypeptide sequence is fused to the C-terminus of CD16 or is positioned internally in the CD16. Typically, up to about 30% of the CD16 cytoplasmic domain may be replaced. Such modification can enhance expression or enhance cytotoxicity (e.g., ADCC responsiveness). In other examples, chimeric proteins, such as domains from other lymphocyte activating receptors, including but not limited to Ig-a, Ig-B, CD3-e, CD3-d, DAP-12 and DAP-10, replace a portion of the CD16 cytoplasmic domain.

Fusion genes can be synthesized by conventional techniques, including automated DNA synthesizers and PCR amplification using anchor primers that give rise to complementary overhangs between two consecutive gene fragments that can subsequently be annealed and re-amplified to generate a chimeric gene sequence (Ausubel, 2002). Many vectors are commercially available that facilitate sub-cloning CD16 in-frame to a fusion moiety.

### Chimeric Antigen Receptor

As described herein, NK-92® cells are further engineered to express a chimeric antigen receptor (CAR) on the cell surface. Optionally, the CAR is specific for a tumor-specific antigen. Tumor-specific antigens are described, by way of non-limiting example, in U.S. 2013/0189268; WO 1999024566 A1; U.S. Pat. No. 7,098,008; and WO 2000020460 A1, each of which is incorporated herein by reference in its entirety. Tumor-specific antigens include, without limitation, NKG2D, CS1, GD2, CD138, EpCAM, EBNA3C, GPA7, CD244, CA-125, ETA, MAGE, CAGE, BAGE, HAGE, LAGE, PAGE, NY-SEO-1, GAGE, CEA, CD52, CD30, MUC5AC, c-Met, EGFR, FAB, WT-1, PSMA, NY-ESO1, AFP, CEA, CTAGIB, CD19 and CD33. Additional non-limiting tumor-associated antigens, and the malignancies associated therewith, can be found in Table 3.

TABLE 3
Tumor-Specific Antigens and Associated Malignancies

| Target Antigen | Associated Malignancy |
|---|---|
| α-Folate Receptor | Ovarian Cancer |
| CAIX | Renal Cell Carcinoma |
| CD19 | B-cell Malignancies |
| | Chronic lymphocytic leukemia (CLL) |
| | B-cell CLL (B-CLL) |
| | Acute lymphoblastic leukemia (ALL); ALL |
| | post Hematopoietic stem cell transplantation |
| | (HSCT) |
| | Lymphoma; Refractory Follicular |
| | Lymphoma; B-cell non-Hodgkin lymphoma |
| | (B-NHL) |
| | Leukemia |
| | B-cell Malignancies post-HSCT |
| | B-lineage Lymphoid Malignancies post |
| | umbilical cord blood transplantation |
| | (UCBT) |
| CD19/CD20 | Lymphoblastic Leukemia |
| CD20 | Lymphomas |
| | B-Cell Malignancies |
| | B-cell Lymphomas |
| | Mantle Cell Lymphoma |
| | Indolent B-NHL |
| | Leukemia |
| CD22 | B-cell Malignancies |
| CD30 | Lymphomas; Hodgkin Lymphoma |
| CD33 | AML |
| CD44v7/8 | Cervical Carcinoma |
| CD138 | Multiple Myeloma |
| CD244 | Neuroblastoma |
| CEA | Breast Cancer |
| | Colorectal Cancer |
| CS1 | Multiple Myeloma |
| EBNA3C | EBV Positive T-cells |
| EGP-2 | Multiple Malignancies |
| EGP-40 | Colorectal Cancer |
| EpCAM | Breast Carcinoma |
| Erb-B2 | Colorectal Cancer |
| | Breast Cancer and Others |
| | Prostate Cancer |
| Erb-B 2,3,4 | Breast Cancer and Others |
| FBP | Ovarian Cancer |
| Fetal Acetylcholine | Rhabdomyosarcoma |
| Receptor | |
| GD2 | Neuroblastoma |
| GD3 | Melanoma |
| GPA7 | Melanoma |
| Her2 | Breast Carcinoma |
| | Ovarian Cancer |
| | Tumors of Epithelial Origin |
| Her2/new | Medulloblastoma |
| | Lung Malignancy |
| | Advanced Osteosarcoma |
| | Glioblastoma |
| IL-13R-a2 | Glioma |
| | Glioblastoma |
| | Medulloblastoma |
| KDR | Tumor Neovasculature |
| k-light chain | B-cell Malignancies |
| | B-NHL, CLL |
| LeY | Carcinomas |
| | Epithelial Derived Tumors |
| L1 Cell Adhesion Molecule | Neuroblastoma |
| MAGE-A1 | Melanoma |
| Mesothelin | Various Tumors |
| MUC1 | Breast Cancer; Ovarian Cancer |
| NKG2D Ligands | Various Tumors |
| Oncofetal Antigen (h5T4) | Various Tumors |
| PSCA | Prostate Carcinoma |
| PSMA | Prostate/Tumor Vasculature |
| TAA Targeted by mAb IgE | Various Tumors |
| TAG-72 | Adenocarcinomas |
| VEGF-R2 | Tumor Neovasculature |

In some embodiments, the CAR targets CD19, CD33 or CSPG-4.

In examples, variant polypeptides are made using methods known in the art such as oligonucleotide-mediated (site-directed) mutagenesis, alanine scanning, and PCR mutagenesis. Site direct mutagenesis (Carter, 1986; Zoller and Smith, 1987), cassette mutagenesis, restriction selection mutagenesis (Wells et al., 1985) or other known techniques can be performed on the cloned DNA to produce CD16 variants (Ausubel, 2002; Sambrook and Russell, 2001).

Optionally, the CAR targets an antigen associated with a specific cancer type.

Optionally, the cancer is selected from the group consisting of leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia), polycythemia vera, lymphomas (e.g., Hodgkin's disease and non-Hodgkin's disease), multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

In some embodiments, a polynucleotide encoding a CAR is mutated to alter the amino acid sequence encoding for CAR without altering the function of the CAR. For example, polynucleotide substitutions leading to amino acid substitutions at “non-essential” amino acid residues can be made in the CARs disclosed above. CARs can be engineered as described, for example, in Patent Publication Nos. WO 2014039523; US20140242701; US20140274909; U.S. Pat. No. 20130280285; and WO 2014099671, each of which is incorporated herein by reference in its entirety. Optionally, the CAR is a CD19 CAR, a CD33 CAR or CSPG-4 CAR.

### Additional Modifications—Cytokines

The cytotoxicity of NK-92® cells is dependent on the presence of cytokines (e.g., interleukin-2 (IL-2). The cost of using exogenously added IL-2 needed to maintain and expand NK-92® cells in commercial scale culture is significant. The administration of IL-2 to human subjects in sufficient quantity to continue activation of NK-92® cells would cause adverse side effects.

In some embodiments, FcR-expressing NK-92® cells are further modified to express at least one cytokine and a suicide gene. In specific embodiments, the at least one cytokine is IL-2, IL-12, IL-15, IL-18, IL-21 or a variant thereof. In preferred embodiments, the cytokine is IL-2. A representative nucleic acid encoding IL-2 is shown in SEQ ID NO:3 and a representative polypeptide of IL-2 is shown in SEQ ID NO:4. In certain embodiments the IL-2 is a variant that is targeted to the endoplasmic reticulum.

In one embodiment, the IL-2 is expressed with a signal sequence that directs the IL-2 to the endoplasmic reticulum. Not to be bound by theory, but directing the IL-2 to the endoplasmic reticulum permits expression of IL-2 at levels sufficient for autocrine activation, but without releasing IL-2 extracellularly. See Konstantinidis et al “Targeting IL-2 to the endoplasmic reticulum confines autocrine growth stimulation to NK-92® cells” Exp Hematol. 2005 February; 33 (2): 159-64. Continuous activation of the FcR-expressing NK-92® cells can be prevented, e.g., by the presence of the suicide gene.

### Additional Modifications—Suicide Genes

The term “suicide gene” is one that allows for the negative selection of the cells. A suicide gene is used as a safety system, allowing the cells expressing the gene to be killed by introduction of a selective agent. This is desirable in case the recombinant gene causes a mutation leading to uncontrolled cell growth. A number of suicide gene systems have been identified, including the herpes simplex virus thymidine kinase (TK) gene, the cytosine deaminase gene, the varicella-zoster virus thymidine kinase gene, the nitroreductase gene, the Escherichia coli gpt gene, and the E. coli Deo gene (also see, for example, Yazawa K, Fisher W E, Brunicardi F C: Current progress in suicide gene therapy for cancer. World J. Surg. 2002 July; 26 (7): 783-9). As used herein, the suicide gene is active in NK-92® cells. Typically, the suicide gene encodes for a protein that has no ill-effect on the cell but, in the presence of a specific compound, will kill the cell. Thus, the suicide gene is typically part of a system.

In one embodiment, the suicide gene is the thymidine kinase (TK) gene. The TK gene may be a wild-type or mutant TK gene (e.g., tk30, tk75, sr39tk). Cells expressing the TK protein can be killed using ganciclovir.

In another embodiment, the suicide gene is Cytosine deaminase which is toxic to cells in the presence of 5-fluorocytosine. Garcia-Sanchez et al. “Cytosine deaminase adenoviral vector and 5-fluorocytosine selectively reduce breast cancer cells 1 million-fold when they contaminate hematopoietic cells: a potential purging method for autologous transplantation.” Blood 1998 Jul. 15; 92 (2): 672-82.

In another embodiment, the suicide gene is cytochrome P450 which is toxic in the presence of ifosfamide, or cyclophosphamide. See e.g., Touati et al. “A suicide gene therapy combining the improvement of cyclophosphamide tumor cytotoxicity and the development of an anti-tumor immune response.” Curr Gene Ther. 2014; 14 (3): 236-46.

In another embodiment, the suicide gene is iCas9. Di Stasi, (2011) “Inducible apoptosis as a safety switch for adoptive cell therapy.” N Engl J Med 365:1673-1683. See also Morgan, “Live and Let Die: A New Suicide Gene Therapy Moves to the Clinic” Molecular Therapy (2012); 20:11-13. The iCas9 protein induces apoptosis in the presence of a small molecule AP1903. AP1903 is biologically inert small molecule, that has been shown in clinical studies to be well tolerated, and has been used in the context of adoptive cell therapy.

In one embodiment, the modified NK-92® cells are irradiated prior to administration to the patient. Irradiation of NK-92® cells is described, for example, in U.S. Pat. No. 8,034,332, which is incorporated herein by reference in its entirety. In one embodiment, modified NK-92® cells that have not been engineered to express a suicide gene are irradiated.

### Transgene Expression

Transgenes (e.g., CD19 CAR and CD16) can be engineered into an expression vector by any mechanism known to those of skill in the art. Transgenes may be engineered into the same expression vector or a different expression vector. In preferred embodiments, the transgenes are engineered into the same vector.

In some embodiments, the vector allows incorporation of the transgene(s) into the genome of the cell. In some embodiments, the vectors have a positive selection marker. Positive selection markers include any genes that allow the cell to grow under conditions that would kill a cell not expressing the gene. Non-limiting examples include antibiotic resistance, e.g., geneticin (Neo gene from Tn5).

Any number of vectors can be used to express the Fc receptor and/or the CAR. In some embodiments, the vector is a plasmid. In one embodiment, the vector is a viral vector. Viral vectors include, but are not limited to, retroviral vectors, adenoviral vectors, adeno-associated viral vectors, herpes simplex viral vectors, pox viral vectors, and others.

Transgenes can be introduced into the NK-92® cells using any transfection method known in the art, including, by way of non-limiting example, infection, electroporation, lipofection, nucleofection, or “gene-gun.”

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### EMBODIMENTS

This disclosure comprises the following, non-limiting embodiments:

### Embodiment 1

A method of freezing NK-92® cells, the method comprising: contacting NK-92® cells with a cell freezing medium, wherein the cell freezing medium comprises a first composition and a second composition, wherein the first composition comprises: (a) one or more electrolytes selected from the group consisting of potassium ions, sodium ions, magnesium ions, and calcium ions; (b) one or more macromolecular agents selected from the group consisting of human albumin, polysaccharide and colloidal starch; (c) a pH buffer; (d) at least one sugar; (e) at least one sugar alcohol; (f) an impermeant agent selected from the group consisting of lactobionate, gluconate, citrate and glycerophosphate; and (g) DMSO; and wherein the second composition comprises albumin.

### Embodiment 2

The method of embodiment 1, wherein the NK-92® cells are contacted with the second composition before adding the first composition.

### Embodiment 3

The method of any of the preceding embodiments, wherein the sugar alcohol is a 6-carbon sugar alcohol.

### Embodiment 4

The method of any of the preceding embodiments, wherein the sugar is a simple sugar.

### Embodiment 5

The method of any of the preceding embodiments, wherein the simple sugar is selected from the group consisting of glucose, dextran, dextrose, trehalose, and maltose.

### Embodiment 6

The method of any of the preceding embodiments, wherein the albumin in the second composition is human albumin.

### Embodiment 7

The method of any of the preceding embodiments, wherein the second composition comprises 2-10% human albumin.

### Embodiment 8

The method of any of the preceding embodiments, wherein the first and second composition are mixed at a volume ratio that ranges from 1:5 to 5:1.

### Embodiment 9

The method of any of the preceding embodiments, wherein first composition comprises 10% DMSO.

### Embodiment 10

The method of any of the preceding embodiments, wherein the first composition further comprises one or more substrates effective for regeneration of ATP

### Embodiment 11

The method of any of the preceding embodiments, wherein the one or more substrates are selected from the group consisting of adenosine, fructose, ribose and adenine.

### Embodiment 12

The method of any of the preceding embodiments, wherein the first composition further comprises glutathione.

### Embodiment 13

The method of any of the preceding embodiments, wherein the first composition further comprises one or more impermeant agents that can maintain ionic and osmotic balance during hypothermia.

### Embodiment 14

The method of any of the preceding embodiments, wherein the first composition and second composition is mixed at a volume ratio between 1:5 and 5:1.

### Embodiment 15

The method of any of the preceding embodiments, wherein a cell freezing medium comprises less than 10% DMSO.

### Embodiment 16

The method of any of the preceding embodiments, wherein the method further comprises freezing the cells using a controlled rate freeze to reach a final temperature of −80° C. or lower.

### Embodiment 17

The method of any of the preceding embodiments, wherein the method further comprises storing the cells at −80° C. to −196° C.

### Embodiment 18

The method of any of the preceding embodiments, wherein the method further comprises thawing the preserved cells.

### Embodiment 19

The method of any of the preceding embodiments, wherein the cells are thawed at 37° C.

### Embodiment 20

The method of any of the preceding embodiments, wherein more than 70% of the cells are viable at the time of thawing.

### Embodiment 21

The method of any of the preceding embodiments, wherein more than 90% of the cells are viable at the time of thawing.

### Embodiment 22

The method of any of the preceding embodiments, wherein the thawed cells have a direct cytoxicity of at least 80% against K562 cells at an effector to target ratio of 10:1.

### Embodiment 23

The method of any of the preceding embodiments, wherein the thawed cells have a direct cytotoxicity that is 70-100% of that of the NK-92® cells before the cells are frozen.

### Embodiment 24

The method of any of the preceding embodiments, wherein the thawed NK-92® cells have a ADCC activity of at least 80-120% against Ramos cells at an effector to target ratio of 10:1, in the presence of an antibody targeting the Ramos cells.

### Embodiment 25

The method of any of the preceding embodiments, wherein the thawed NK-92® cells have a ADCC activity that is 80-100% of that of the cells before the cells are frozen.

### Embodiment 26

The method of any of the preceding embodiments, wherein the method further comprises administering the thawed cells to a patient in need via infusion.

### Embodiment 27

The method of any of the preceding embodiments, wherein the NK-92® cells express a cytokine, Fc Receptor, chimeric antigen receptor, or a combination thereof.

### Embodiment 28

A NK-92® cell culture comprising: NK-92® cells and a cell freezing medium, wherein the cell freezing medium comprises a first composition and a second composition, wherein the first composition comprises: (a) one or more electrolytes selected from the group consisting of potassium ions, sodium ions, magnesium ions, and calcium ions; (b) one or more macromolecular agents selected from the group consisting of human albumin, polysaccharide and colloidal starch; (c) a pH buffer; (d) at least one sugar; (e) at least one sugar alcohol; (f) an impermeant agent being at least one member selected from the group consisting of lactobionate, gluconate, citrate and glycerophosphate; and (g) DMSO; and wherein the second composition comprises albumin.

### Embodiment 29

The NK-92® cell culture of embodiment 28, wherein the sugar alcohol is a 6-carbon sugar alcohol.

### Embodiment 30

The NK-92® cell culture of any of the preceding embodiments, wherein the sugar is a simple sugar.

### Embodiment 31

The NK-92® cell culture of any of the preceding embodiments, wherein the simple sugar is selected from the group consisting of glucose, dextran, dextrose, trehalose, and maltose.

### Embodiment 32

The NK-92® cell culture of any of the preceding embodiments, wherein the second composition is human albumin.

### Embodiment 33

The NK-92® cell culture of any of the preceding embodiments, wherein the second composition is 2-10% human albumin.

### Embodiment 34

The NK-92® cell culture of any of the preceding embodiments, wherein the first composition and second composition is mixed at a volume ratio ranging from 5:1 to 1:5.

### Embodiment 35

The NK-92® cell culture of any of the preceding embodiments, wherein the first composition comprises 10% DMSO.

### Embodiment 36

The NK-92® cell culture of any of the preceding embodiments, wherein the first composition further comprises a substrate effective for the regeneration of ATP.

### Embodiment 37

The NK-92® cell culture of any of the preceding embodiments, wherein the substrate in the first composition is selected from the group consisting of adenosine, fructose, ribose and adenine.

### Embodiment 38

The NK-92® cell culture of any of the preceding embodiments, wherein the first composition comprises 20-60 mM potassium ions.

### Embodiment 39

The NK-92® cell culture of any of the preceding embodiments, wherein the first composition comprises 50-150 mM sodium ions.

### Embodiment 40

The NK-92® cell culture of any of the preceding embodiments, wherein the first composition comprises 0.001-1 mM magnesium ions.

### Embodiment 41

The NK-92® cell culture of any of the preceding embodiments, wherein the first composition further comprises glutathione.

### Embodiment 42

The NK-92® cell culture of any of the preceding embodiments, wherein the cell culture is kept at 80° C. to −196° C.

### Embodiment 43

The NK-92® cell culture of any of the preceding embodiments, wherein at least 70% of NK-92® cells in the cell culture that has been kept at −80° C. to −196° C. are viable after cell culture is thawed.

### Embodiment 44

The NK-92® cell culture of any of any of the preceding embodiments, wherein the NK-92® cells comprise a cytokine, Fc Receptor, chimeric antigen receptor, or a combination thereof.

### Embodiment 45

A cell freezing medium produced by mixing a first composition and a second composition at a one to one ratio, wherein the first composition comprises: (a) one or more electrolytes selected from the group consisting of potassium ions, sodium ions, magnesium ions, and calcium ions; (b) one or more macromolecular agents selected from the group consisting of human albumin, polysaccharide and colloidal starch; (c) a pH buffer; (d) at least one sugar; (e) at least one sugar alcohol; (f) an impermeant agent selected from the group consisting of lactobionate, gluconate, citrate and glycerophosphate; and (g) DMSO; and wherein the second composition comprises albumin.

### Embodiment 46

A method of harvesting NK-92® cells comprising collecting NK-92® cells from a cell culture and washing the collected NK-92® cells by resuspending the cells in a buffer comprising 1-5% albumin.

### Embodiment 47

The method of embodiment 46, wherein the collecting NK-92® cells comprising centrifuging the NK-92® cells in the cell culture.

### Embodiment 48

The method of any of the preceding embodiments, further comprising placing the washed NK-92® cells in an infusion bag.

### Embodiment 49

The method of any of the preceding embodiments, wherein the wash is performed by centrifuging the cells and then resuspending the cells in the wash buffer.

### Embodiment 50

The method of any of the preceding embodiments, wherein the wash is performed at least three times.

### Embodiment 51

The method of any of the preceding embodiments, wherein the wash is performed 4-6 times.

### Embodiment 52

The method of any of the preceding embodiments, wherein the method recovers at least 80% of the NK-92® cells.

### Embodiment 53

The method of any of the preceding embodiments, wherein the viability of the harvested cells is at least 90%.

### Embodiment 54

The method of any of the preceding embodiments, wherein the NK-92® cells that have been harvested have substantially the same cytotoxicity and/or viability as control NK-92® cells that have not been harvested.

### Embodiment 55

The method of any of the preceding embodiments, wherein the NK-92® cells that have been harvested have substantially the same cytotoxicity and/or viability as the NK-92® cells before harvesting.

### Embodiment 56

The method of any of the preceding embodiments, wherein the NK-92® cells that have been harvested have a cytotoxicity of 80-100% on K562 cells.

### Embodiment 57

The method of any of the preceding embodiments, wherein the buffer contains 2-5% albumin.

### Embodiment 58

The method of any of the preceding embodiments, wherein the buffer contains 3-5% albumin.

### Embodiment 59

The method of any of the preceding embodiments, wherein the buffer contains 5% albumin.

### Embodiment 60

The method of any of the preceding embodiments, wherein the buffer lacks sugar.

### Embodiment 61

The method of any of the preceding embodiments, wherein the buffer lacks dextran.

### Embodiment 62

The method of any of the preceding embodiments, wherein the centrifugation is by continuous centrifugation.

### Embodiment 63

The method of any of the preceding embodiments, wherein the albumin is human albumin, or human albumin derived from serum, or human albumin derived from plasma.

### Embodiment 64

The method of any of the preceding embodiments, wherein the NK-92® cells express a cytokine, Fc Receptor, a chimeric antigen receptor, or a combination thereof.

### Embodiment 65

The method of any of the preceding embodiments, wherein the chimeric antigen receptor is a receptor for HER2, CD19 or PD-L1.

### Embodiment 66

The method of any of the preceding embodiments, wherein the chimeric antigen receptor is a receptor for any of the tumor specific antigens listed in Table 2.

### EXAMPLES

The following examples are for illustrative purposes only and should not be interpreted as limitations. There are a variety of alternative techniques and procedures available to those of skill in the art which would similarly permit one to successfully perform the examples below.

### Example 1: NK-92® Cell Formulation, Freeze and Thaw Procedures

NK-92® cells were expanded in Xuri bioreactors, harvested and concentrated using kSep400 centrifuge. Concentrated cells were resuspended in the cell freezing medium, which is a mixture of 5% HA and CryoStor®10 (CS10), which comprises the components as listed in Table 1, at a volume ratio of 1:1, for cryopreservation. Formulated cells are transferred to the Cell Freeze® cryobags (CF-750 supplied by Charter Medical Limited, Winston-Salem, North Carolina) at the proposed clinical dose (2×10e7 cells/mL, 100 mL volume). Cell Freeze® cryobags were subsequently frozen using a CryoMed controlled rate freezer using a preset freezing Profile 4. Frozen bags were stored in vapor phase of a LN2 cryofreezer (≤120° C.). Frozen cells were removed from the LN2 freezer when ready to thaw for infusion. Cryofreeze bags were transferred immediately to a water bath containing a beaker with water at 37° C.

### Example 2: Viability of the NK-92® Cells After Thawing

After thaw, the cells from each bag/vial were counted and the total viable cells collected was calculated. High cell viability (>90%) was observed at thaw. Viability of the cells were determined and shown in the Table 4 below. A fresh NK-92® cell sample in the same cell freezing medium before freezing was used as control (“Pre-freeze”) and counted and viability checked. (Note that the NK-92® cells used in Example 2 are aNK™ cells.)

TABLE 4
Viability Data

| | % Viability at | % Viability |
|---|---|---|
| Experiment | Pre-freeze | at thaw |
| #1 | 96.3% | 91.2% |
| #2 | 96.8% | 95.2% |
| #3 | 94% | 90% |
| #4 | 95.3% | 91% |
| #5 | 96.5% | 95.1% |
| #6 | 95.9% | 95.3% |

### Example 3: Phenotypes of the NK-92® Cells After Thawing

A study was conducted to quantify the expression of a panel of six surface protein markers on the NK-92® cells that has been thawed as described in Example 1. The expression profile is compared to the profile of the NK-92® cells in the control sample. The panel of surface markers was selected to be representative of natural killer (NK) cells. The surface markers CD54, CD56, NKG2D, NKp30, CD3, and CD16 were analyzed and the marker expression was determined by staining cells with specific fluorochrome-conjugated antibodies and detecting bound antibodies by flow cytometry.

The results show that expression of surface markers (CD56, CD54, CD3, CD16, NKG2D and NKp30) on NK-92® cells (aNK™ cells or haNK® cells) was comparable between the control samples (fresh samples) and those that had been cryopreserved using CryoStor®10 (CS10)+HA Surface marker expression on NK-92® cells were analyzed by flow cytometry-based staining and the results are presented in Table 5 below.

TABLE 5
Phenotypes of Cryopreserved NK-92 ® cells at thaw

| | Description | CD3 | CD56 | CD16 | CD54 | NKG2D | NKp30 |
|---|---|---|---|---|---|---|---|
| Experiment #1 | aNK ™ cells at Pre-freeze | 0.90 | 99.90 | 4.50 | 99.70 | 98.50 | 98.50 |
| | aNK ™ cells | 0.30 | 99.70 | 1.30 | 99.80 | 99.00 | 95.80 |
| | post thaw | | | | | | |
| Experiment #2 | haNK ® cells at Pre-freeze | 0.11 | 99.98 | 98.82 | 99.95 | 83.03 | 99.16 |
| | haNK ® cells post thaw | 0.34 | 99.88 | 98.68 | 99.94 | 80.32 | 99.66 |
| Experiment # | haNK ® cells at Pre-freeze | 0.07 | 99.7 | 97.73 | 99.82 | 90.41 | 99.39 |
| | haNK ® cells post thaw | 0.28 | 99.82 | 98.57 | 99.91 | 89.48 | 99.83 |

### Example 4: Cytotoxicity of the NK-92® Cells After Thawing

Direct cytotoxicity of the thawed aNK™ cells after cryopreservation was evaluated against K562 cell line. aNK™ cells were mixed with the calcein loaded target cells at the Effector: Target ratio of 10:1. Calcein release was assessed by fluorescence plate reader post 3 hours of co-incubation. The results are shown in Table 6 below.

TABLE 6
Direct Cytotoxicity

| Description | % Cytotoxicity |
|---|---|
| aNK ™ at Pre-freeze | 97 ± 5 |
| aNK ™ at thaw | 88 ± 1 |

Comparison of the control samples with cryopreserved test samples showed that cryopreserved NK-92® cells retained their direct cytotoxicity of K562 target cells at thaw; the percent of direct cytotoxicity was shown as 88±1%.

haNK® cells were mixed with the calcein labelled Ramos cells (target cells) in the presence of Rituxan antibody, which targets the CD20 expressed on target cells, at the Effector: Target ratio of 10:1. The anti β-gal antibody was used as a control for cytotoxicity not related to ADCC. Calcein release was assessed by fluorescence plate reader post 3 hour of co-incubation. Cytotoxicity was determined using a Calcein-release assay and is expressed as the percentage (%) of Calcein release at an Effector: Target ratio of 10:1 (mean±standard deviation). Each sample was assayed in triplicate. The results are shown in Table 7.

TABLE 7
ADCC

| | | % Specific lysis | |
|---|---|---|---|
| Experiment | Sample | Rituxan | Anti β-gal |
| #1 | Pre-Freeze | 107 ± 4 | 13 ± 1 |
| | Post-Thaw | 99 ± 2 | 12 ± 2 |
| #2 | Pre-Freeze | 106 ± 5 | 16 ± 1 |
| | Post-Thaw | 95 ± 4 | 14 ± 1 |

The results show that the cells that have been frozen and thawed (post-thaw state) as described retains ADCC activity, e.g., 99±2% or 95±4%. And the ADCC activity of the cells at post-thaw state is substantially the same as, e.g., about 92.5% (99/107), or 90% (95/106) of the ADCC activity of the cells at the pre-freeze state.

### Example 5: Washing Cells Using 5% Of Human Albumin Increases Efficiency Of Cell Havesting

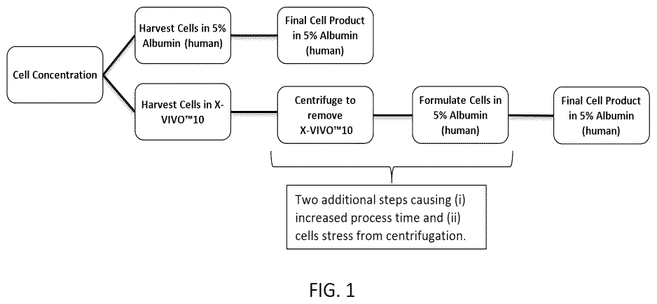

FIG. 1 shows the processes of harvesting modified NK-92® (HER2·taNK) cells from large bioreactors using either X-VIVO™M10 or 5% HA (human albumin). Although cells can be concentrated using both methods, harvesting in X-VIVO™M10 requires two additional steps that result in increased process time and cell stress and loss due to centrifugation. Harvesting cells in 5% HUMAN ALBUMIN simplifies the process and improves harvesting efficiency.

### Example 6: NK-92® Cell Viability After Being Thawed in 5% Human Albumin

Frozen modified NK-92® (HER2·taNK) cells were thawed in a 37° C. water bath. 100 μL of the thawed cells were added to 900 μL of the X-VIVO™10 medium, 5% Human Albumin, and PBS, respectively. Cell viability was measured using a Nucleocounter NC-200™ and shown in Table 8.

TABLE 8
Cell viability after being thawed in 5% HUMAN ALBUMIN

| | | | % |
|---|---|---|---|
| Experiment | Study Number | Thaw Media | Viability |
| Thaw of | NKSTUDYTP_028 | X-VIVO ™10 | 94.4 |
| Frozen | | 5% Human Albumin | 86.5 |
| HER2.taNK cells | | PBS | 74.6 |

The results show that cells thawed in 5% Human Albumin had 86.5% viability, which although not as high as X-VIVO™10 (94.4%), was significantly higher than the viability of cells thawed in PBS (74.6%). Cells thawed in 5% Human Albumin and cells thawed in X-VIVO™10 were transferred to respective G-Rex flasks in growth media for expansion, and further expanded in 25 L growth media and 10 L growth media in Xuri bags, respectively.

### Example 7: Cytotoxicity of Modified NK-92® (HER2·taNK) Cells on BT-474 Target Cells

Modified NK-92® (HER2·taNK) cells grown in Xuri bags were collected and washed with either X-VIVO™M10 medium or 5% Human Albumin using continuous centrifugation. For each group, cytotoxicity assays were performed on cells that were used to feed the continuous centrifuge (i.e., cells that had not undergone harvesting process, referred to as the pre-harvest sample); cells that exit the continuous centrifuge (i.e., cells that had completed the harvesting process, referred to as the post-harvest sample); and cells from the G-rex flask, which had not undergone the harvesting process and were served as control cells. To assess cytotoxicity, cells were mixed with the calcein-loaded target cells at the Effector: Target ratio of 10:1. Calcein release was assessed by fluorescence plate reader post 3 hours of co-incubation. Cytotoxicity was determined using a calcein-release assay and was expressed as the mean±standard deviation percentage (%) of calcein release. The Effector: Target ratio was 10:1 and samples were assayed in triplicates.

The results show that the cytotoxicity of the modified NK-92® (HER2·taNK) cells harvested using 5% Human Albumin as wash buffer, which was 88±10%, was substantially the same as that of HER2·taNK cells that were harvested using X-VIVO™ 10 (90±4%). The cytotoxicity of the HER2·taNK cells harvested using 5% Human Albumin was also substantially the same as the cytotoxicity of the control HER2·taNK cells, i.e., the cells in the G-Rex flask, which was 90±5%. Further, using 5% Human Albumin as cell wash medium did not impair cytotoxicity of the HER2·taNK cells, as reflected in that the cytotoxicity of the pre-harvest sample and the cytotoxicity of the post-harvest sample were also substantially similar. See Table 9.

TABLE 9
Cytotoxicity of HER2.taNK cells on BT-474 target cells

| | Wash and | | % |
|---|---|---|---|
| Experiment | Harvest Media | Test Sample | Cytotoxicity |
| HER2.taNK | X-VIVO ™ 10 | Pre-harvest | 89 ± 2 |
| cells vs | | Post-harvest | 90 ± 4 |
| BT-474 cells | | G-Rex (reference | 89 ± 1 |
| | | control) | |
| HER2.taNK | 5% Albumin | Pre-harvest | 86 ± 4 |
| cells vs | (Human) | Post-harvest | 88 ± 10 |
| BT-474 cells | | G-Rex (reference | 90 ± 5 |
| | | control) | |

The results show that the cytotoxicity of the modified NK-92® (CD19 t-haNK and PD-L1 t-haNK) cells harvested using 5% Albumin (Human) as wash buffer (post-harvest), against tumor cells of different origin were comparable to the cytotoxicity of the cells taken directly from bioreactor (pre-harvest) as well as the reference control cells i.e., the cells in the G-Rex flask. See Table 10.

TABLE 10
Cytotoxicity of modified NK-92 ® cells against tumor cells of different origin

| Modified NK-92 ® | | | | % |
|---|---|---|---|---|
| Effector Cells | Tumor Type | Study Date | Test Sample | Cytotoxicity¹ |
| CD19 t-haNK | B Cell Lymphoma | NKSTUDYTP_100 | Reference | 92 ± 7 |
| Cells | SUP-B15 cells | 13 Jul. 2018 | Pre-harvest | 102 ± 7 |
| | | | Post-harvest | 99 ± 3 |
| | Leukemia Cells | NKSTUDYTP_100 | Reference | 86 ± 7 |
| | K562 cells | 13 Jul. 2018 | Pre-harvest | 72 ± 8 |
| | | | Post-harvest | 81 ± 5 |
| PD-L1 t-haNK | Breast Tumor Cells | NKSTUDYTP_095 | Reference | 87 ± 3 |
| Cells | MDA-MB-231 cells | 11 Jul. 2018 | Pre-harvest | 87 ± 1 |
| | (Triple Negative) | | Post-harvest | 86 ± 4 |

¹ModifiedNK-92 ® cells were centrifuged and harvested in 5% HA. Cells were mixed with the calcein loaded target cells of different origins and target lysis was evaluated by measuring Calcein release in the culture media. Cells from G-Rex were used as reference control.

The results show that the modified NK-92® (haNK, and CD19 t-haNK, and PD-L1 t-haNK) cells harvested using 5% Albumin (Human) as wash buffer (post-harvest), induced effective antibody dependent cellular cytotoxicity (ADCC) of tumor cells when coupled with different therapeutic antibodies of clinical grade. Comparable ADCC was observed between reference, pre-harvest and post-harvest samples. See Table 11.

TABLE 11
ADCC Activity of Modified NK-92 ® (haNK, CD19
t-haNK and PD-L1 t-haNK) cells against tumor cells
in combination with therapeutic antibodies of clinical grade

| Modified NK- | | | | |
|---|---|---|---|---|
| 92 ® Effector | | Study Number | Test | % |
| Cells | Tumor Cells | and Date | Sample | Cytotoxicity¹ |
| haNK cells | Ramos | NKSTUDYTP_058 | Reference | 101 ± 5 |
| | cells + | 16 May 2018 | Pre-harvest | 97 ± 2 |
| | Rituxan | | Post-harvest | 93 ± 1 |
| CD19 t-haNK | MDA-MB-231 | NKSTUDYTP_101 | Reference | 50 ± 9 |
| Cells | cells + | 31 Jul. 2018 | Pre-harvest | 42 ± 2 |
| | Avelumab | | Post-harvest | 45 ± 2 |
| PD-L1 t-haNK | MDA-MB-453 | NKSTUDYTP_103 | Reference | 58 ± 1 |
| Cells | cells + | 17 Aug. 2018 | Pre-harvest | 61 ± 2 |
| | Herceptin | | Post-harvest | 70 ± 3 |

¹Modified NK-92 ® cells were centrifuged and harvested in 5% HA. Cells were mixed with the calcein loaded target cells of different origins in the presence of therapeutic antibodies and the target lysis was evaluated by measuring Calcein release in the culture media. Cells from G-Rex were used as reference control.

### Example 8: Viability and Recovery of NK-92® Cells Harvested from Continuous Centrifugation in 5% Albumin (Human)

Modified NK-92® (HER2·taNK) cells were harvested using continuous centrifugation in either X-VIVO™ 10 or 5% Albumin (Human). Cell viability of pre-harvest and post-harvest samples was examined using a Nucleocounter NC-200 cell counting method and a trypan blue dye-exclusion method. Percent recovery was calculated as the amount of cells recovered from continuous centrifugation/amount of cells entered into continuous centrifugation. The results show that viability of cells harvested from either X-VIVO™ 10 or 5% Albumin (Human) were comparable, 96.7% versus 95.7%. Further, percent cell recovery when harvested using 5% Albumin (Human) was 91.9%, slightly higher than the harvesting using X-VIVO™ 10, which was 89.9%. Further, the viability of the pre-harvest sample and the post-harvest sample was also comparable, indicating that washing cells with 5% Albumin (Human) did not impair cell viability. See Table 12.

TABLE 12
Viability And Recovery Of the Harvested Modified
NK-92 ® (HER2.taNK) Cells

| | | | | % |
|---|---|---|---|---|
| Harvest | Study Date & | Test | % | Re- |
| Media | Number | Sample | Viability | covery |
| X-VIVO ™10 | 22 Jun. 2017 | Pre-harvest | 96.6 | 89.9 |
| | NKSTUDYPRT006 | Post-harvest | 96.7 | |
| 5% ALBUMIN | 27 Jun. 2017 | Pre-harvest | 98 | 91.9 |
| (HUMAN) | NKSTUDYPRT006 | Post-harvest | 95.7 | |

### Example 9: Surface Expression of Modified NK-92® (CD19 t-haNK) Cells Harvested from Continuous Centrifugation in 5% Albumin (Human)

Modified NK-92® cells were harvested using continuous centrifugation in 5% Albumin (Human) and the surface expression of the samples were examined using Flow Cytometry based method. Percent surface marker expression was not affected by 5% Albumin (Human) wash as similar amounts of expression was observed on cells from post-harvest, pre-harvest as well as reference cells. See Tables 13 and 14.

TABLE 8
Phenotyping of the Harvested CD19 t-haNK Cells

| Study Date & | | Cell Surface Marker (%)¹ | | | | | |
|---|---|---|---|---|---|---|---|
| Number | Test Sample | CD3 | CD56 | CD16 | CD54 | NKG2D | NKp30 |
| NKSTUDYTP_100 | Reference | 0.1 | 100 | 99 | 100 | 100 | 99 |
| 20 Jul. 2018 | Pre-harvest | 0.0 | 100 | 96 | 100 | 100 | 98 |
| | Post-harvest | 0.0 | 100 | 95 | 100 | 99 | 95 |

¹Percent (%) of CD19 t-haNK cells positive for the cell surface marker by Flow Cytometry

TABLE 14
CAR-expression on the Harvested Modified
NK-92 ® (CD19 t-haNK) Cells

| Study Date & Number | Test Sample | % CAR+¹ |
|---|---|---|
| NKSTUDYTP_100 | Reference | 99 |
| 20 Jul. 2018 | Pre-harvest | 99 |
| | Post-harvest | 99 |

¹Percent of CD19 t-haNK cells positive for CD19.CAR expression determined by Flow Cytometry

### Example 10: Safe Administration of Processed Modified NK Cells Into Human Patients

Modified NK cells prepared according to methods disclosed herein were administered to human patients. As disclosed in the following publications and clinical trials, vaccines that contain the modified NK cells were safely administered to patients. The content of each of the following publications is incorporated herein by reference.
(1) Moini et al., The Oncologist, 2025 (hereinafter “Moini”);
(2) Seery et al., Gastrointestinal Cancers Symposium, 2019 (hereinafter “Seery 1”);
(3) Seery et al., American Society of Clinical Oncology Gastrointestinal Cancers (ASCO GI) Symposium, 2023 (hereinafter “Seery 2”);
(4) ClinicalTrials.gov ID NCT03586869; and
(5) ClinicalTrials.gov ID NCT04390399.

As disclosed in Seery 1, a pancreatic cancer vaccine comprising immortalized, modified NK cells was administered to human patients enrolled in clinical trial NCT03586869. The immortalized NK cells (“haNK” cells) were modified to express the high affinity CD16 V158 FcγRIIIa receptor and IL-2. The safety and efficacy of the vaccine were evaluated in the clinical trial. Seery 1 discloses that the clinical trial showed that the vaccine comprising the immortalized, modified NK cells “could be safely administered in an outpatient setting, with [adverse effects] that were manageable by dose-reduction, and preliminary survival results that exceed the standard of care.” Thus, cells prepared according to the methods disclosed herein were safely administered into human patients.

Seery 2 and Moini also disclose a pancreatic cancer vaccine comprising immortalized, modified NK cells (“PD-L1 t-haNK” cells) that express high-affinity CD16 for binding to PD-L1. This vaccine was also administered to human patients in clinical trial NCT04390399. Seery 2 discloses that “100% of treatments were performed in the outpatient setting with acceptable toxicity” and that the median overall survival (OS) had doubled when compared to historical OS for patients that did not receive the vaccine comprising the immortalized, modified NK cells. Further, Moini concluded that its vaccine comprising the immortalized, modified NK cells “prevented disease progression and supported the patient's continued survival.” Thus, again, cells prepared according to the methods disclosed herein were safely administered into human patients.

The cells in Seery 1, clinical trial NCT03586869, Seery 2, Moini, and clinical trial NCT04390399 were prepared according to the following methods. In detail, the immortalized, modified NK cells were first produced in a continuous culture process. To initiate a culture for manufacture of a drug substance, a cell vial was thawed into growth medium. The thawed cells were cultured in a T-flask and the cell number was expanded through serial passaging in G-Rex flasks containing growth medium (GM). The culture was maintained in G-Rex flasks prior to scale-up expansion in the Xuri™ Cell Expansion System W25 (Cytiva™) followed by Xcellerex™ XDR-200 stirred-tank bioreactor (Cytiva™). The expanded immortalized, modified NK cells were harvested using kSep400. During the Ksep® 400 (Sartorius) centrifugation concentration process, the cells were washed multiple times with 5% human albumin and were eluted into a transfer flask. As they were, the cells directly administered to human patients as a vaccine product.

In some cases, it was beneficial to freeze the cells to extend shelf-life or to preserve the biological state of the cells during shipping. After the albumin wash step described in the preceding paragraph was performed, the washed cells were irradiated using a gamma-ray source per clinical practice standards, and then mixed with cryopreservation medium at 1:1 to prepare the vaccine product. The vaccine comprising the prepared cells were then frozen for long-term storage, and subsequently thawed for direct administration into patients without further manipulation.

The above-referenced disclosures provide evidence that the immortalized, modified NK cells prepared according to the methods disclosed herein were readily infused to human patients. Regardless of whether the cells were frozen and thawed or not, the cells prepared according to the methods disclosed herein were indeed administered human patients as a ready-to-use vaccine product.

```
Informal Sequence Listing
High Affinity Variant Immunoglobulin Gamma Fc Region Receptor III-
A nucleic acid sequence (full length form).
SEQ ID NO: 1
ATGTGGCA GCTGCTGCTG CCTACAGCTC TCCTGCTGCT GGTGTCCGCC
GGCATGAGAA CCGAGGATCT GCCTAAGGCC GTGGTGTTCC TGGAACCCCA
GTGGTACAGA GTGCTGGAAA AGGACAGCGT GACCCTGAAG TGCCAGGGCG
CCTACAGCCC CGAGGACAAT AGCACCCAGT GGTTCCACAA CGAGAGCCTG
ATCAGCAGCC AGGCCAGCAG CTACTTCATCGACGCCGCCA CCGTGGACGA
CAGCGGCGAG TATAGATGCC AGACCAACCT GAGCACCCTGAGCGACCCCG
TGCAGCTGGA AGTGCACATC GGATGGCTGC TGCTGCAGGC
CCCCAGATGGGTGTTCAAAG AAGAGGACCC CATCCACCTG AGATGCCACT
CTTGGAAGAA CACCGCCCTGCACAAAGTGA CCTACCTGCA GAACGGCAAG
GGCAGAAAGT ACTTCCACCA CAACAGCGAC TTCTACATCC CCAAGGCCAC
CCTGAAGGAC TCCGGCTCCT ACTTCTGCAG AGGCCTCGTGGGCAGCAAGA
ACGTGTCCAG CGAGACAGTG AACATCACCA TCACCCAGGG
CCTGGCCGTGTCTACCATCA GCAGCTTTTT CCCACCCGGC TACCAGGTGT
CCTTCTGCCT CGTGATGGTGCTGCTGTTCG CCGTGGACAC CGGCCTGTAC
TTCAGCGTGA AAACAAACAT CAGAAGCAGCACCCGGGACT GGAAGGACCA
CAAGTTCAAG TGGCGGAAGG ACCCCCAGGA CAAGTGA
High Affinity Variant Immunoglobulin Gamma Fc Region Receptor III-
A amino acid sequence (full length form). The Val at position 176 is underlined.
SEQ ID NO: 2
Met Trp Gln Leu Leu Leu Pro Thr Ala Leu Leu Leu Leu Val Ser Ala Gly Met Arg Thr Glu
Asp Leu Pro Lys Ala Val Val Phe Leu Glu Pro Gln Trp Tyr Arg Val Leu Glu Lys Asp Ser
Val Thr Leu Lys Cys Gln Gly Ala Tyr Ser Pro Glu Asp Asn Ser Thr Gln Trp Phe His Asn
Glu Ser Leu Ile Ser Ser Gln Ala Ser Ser Tyr Phe Ile Asp Ala Ala Thr Val Asp Asp Ser Gly
Glu Tyr Arg Cys Gln Thr Asn Leu Ser Thr Leu Ser Asp Pro Val Gln Leu Glu Val His Ile Gly
Trp Leu Leu Leu Gln Ala Pro Arg Trp Val Phe Lys Glu Glu Asp Pro Ile His Leu Arg Cys
His Ser Trp Lys Asn Thr Ala Leu His Lys Val Thr Tyr Leu Gln Asn Gly Lys Gly Arg Lys
Tyr Phe His His Asn Ser Asp Phe Tyr Ile Pro Lys Ala Thr Leu Lys Asp Ser Gly Ser Tyr Phe
Cys Arg Gly Leu Val Gly Ser Lys Asn Val Ser Ser Glu Thr Val Asn Ile Thr Ile Thr Gln Gly
Leu Ala Val Ser Thr Ile Ser Ser Phe Phe Pro Pro Gly Tyr Gln Val Ser Phe Cys Leu Val Met
Val Leu Leu Phe Ala Val Asp Thr Gly Leu Tyr Phe Ser Val Lys Thr Asn Ile Arg Ser Ser Thr
Arg Asp Trp Lys Asp His Lys Phe Lys Trp Arg Lys Asp Pro Gln Asp Lys
ER IL-2 nucleic acid sequence
SEQ ID NO: 3
ATGTACCGGATG CAGCTGCTGA GCTGTATCGC CCTGTCTCTG GCCCTCGTGA
CCAACAGCGC CCCTACCAGC AGCAGCACCA AGAAAACCCA GCTGCAGCTG
GAACATCTGC TGCTGGACCTGCAGATGATC CTGAACGGCA TCAACAACTA
CAAGAACCCC AAGCTGACCC GGATGCTGACCTTCAAGTTC TACATGCCCA
AGAAGGCCAC CGAACTGAAA CATCTGCAGT GCCTGGAAGAGGAACTGAAG
CCCCTGGAAG AAGTGCTGAA CCTGGCCCAG AGCAAGAACT
TCCACCTGAGGCCCAGGGAC CTGATCAGCA ACATCAACGT GATCGTGCTG
GAACTGAAAG GCAGCGAGACAACCTTCATG TGCGAGTACG CCGACGAGAC
AGCTACCATC GTGGAATTTC TGAACCGGTGGATCACCTTC TGCCAGAGCA
TCATCAGCAC CCTGACCGGC TCCGAGAAGG ACGAGCTGTGA
ER IL-2 (ER retention signal is underlined) amino acid sequence
SEQ ID NO: 4
Met Tyr Arg Met Gln Leu Leu Ser Cys Ile Ala Leu Ser Leu Ala Leu Val Thr Asn Ser Ala Pro
Thr Ser Ser Ser Thr Lys Lys Thr Gln Leu Gln Leu Glu His Leu Leu Leu Asp Leu Gln Met Ile
Leu Asn Gly Ile Asn Asn Tyr Lys Asn Pro Lys Leu Thr Arg Met Leu Thr Phe Lys Phe Tyr
Met Pro Lys Lys Ala Thr Glu Leu Lys His Leu Gln Cys Leu Glu Glu Glu Leu Lys Pro Leu
Glu Glu Val Leu Asn Leu Ala Gln Ser Lys Asn Phe His Leu Arg Pro Arg Asp Leu Ile Ser
Asn Ile Asn Val Ile Val Leu Glu Leu Lys Gly Ser Glu Thr Thr Phe Met Cys Glu Tyr Ala Asp
Glu Thr Ala Thr Ile Val Glu Phe Leu Asn Arg Trp Ile Thr Phe Cys Gln Ser Ile Ile Ser Thr
Leu Thr Gly Ser Glu Lys Asp Glu Leu
the amino acid sequence of PD-Ll CAR
SEQ ID NO: 5
    1 MDWIWRILFL VGAATGAHSA QPANIQMTQS PSSVSASVGD RVTITCRASQ DISRWLAWYQ
  61 QKPGKAPKLL IYAASSLQSG VPSRFSGSGS GTDFALTISS LQPEDFATYY CQQADSRFSI
121 TFGQGTRLEI KGGGGSGGGG SGGGGSGGGG SEVQLVQSGG GLVQPGGSLR LSCAASGFTF
181 SSYSMNWVRQ APGKGLEWVS YISSSSSTIQ YADSVKGRFT ISRDNAKNSL YLQMNSLRDE
241 DTAVYYCARG DYYYGMDVWG QGTTVTVSSA AALSNSIMYF SHFVPVFLPA KPTTTPAPRP
301 PTPAPTIASQ PLSLRPEACR PAAGGAVHTR GLDFACFWVL VVVGGVLACY SLLVTVAFII
361 FWVRLKIQVR KAAITSYEKS DGVYTGLSTR NQETYETLKH EKPPQ
the amino acid sequence of CD19 CAR
SEQ ID NO: 6
    1 MDWIWRILFL VGAATGAHSA QPADIQMTQT TSSLSASLGD RVTISCRASQ DISKYLNWYQ
  61 QKPDGTVKLL IYHTSRLHSG VPSRFSGSGS GTDYSLTISN LEQEDIATYF CQQGNTLPYT
121 FGGGTKLELK RGGGGSGGGG SGGGGSGGGG SEVQLQQSGP GLVAPSQSLS VTCTVSGVSL
181 PDYGVSWIRQ PPRKGLEWLG VIWGSETTYY NSALKSRLTI IKDNSKSQVF LKMNSLQTDD
241 TAIYYCAKHY YYGGSYAMDY WGQGTTVTVS SAAALSNSIM YFSHFVPVFL PAKPTTTPAP
301 RPPTPAPTIA SQPLSLRPEA CRPAAGGAVH TRGLDFACFW VLVVVGGVLA CYSLLVTVAF
361 IIFWVRLKIQ VRKAAITSYE KSDGVYTGLS TRNQETYETL KHEKPPQ
the amino acid sequence of Her2 CAR
SEQ ID NO: 7
MDWIWRILFLVGAATGAHSAQPADIQMTQSPSSLSASVGDRVTITCRASQDVNTAVAWYQQK
PGKAPKLLIYSASFLYSGVPSRFSGSRSGTDFTLTISSLQPEDFATYYCQQHYTTPPTFGQG
TKVEIKSSGGGGSGGGGSGGGGSGGGGSGGSEVQLVESGGGLVQPGGSLRLSCAASGFNIKD
TYIHWVRQAPGKGLEWVARTYPTNGYTRYADSVKGRFTISADTSKNTAYLQMNSLRAEDTAV
YYCSRWGGDGFYAMDYWGQGTLVTVSS
```

1. A method of freezing cells from an immortalized non-Hodgkin's lymphoma derived cytolytic cancer cell line (CRL-2407), the method comprising:
contacting the cells with a cell freezing medium, wherein the cell freezing medium comprises a first composition and a second composition, wherein the first composition comprises:
(a) one or more electrolytes selected from the group consisting of potassium ions, sodium ions, magnesium ions, and calcium ions;
(b) one or more macromolecular agents selected from the group consisting of human albumin, polysaccharide and colloidal starch;
(c) a pH buffer;
(d) at least one sugar;
(e) at least one sugar alcohol;
(f) an impermeant agent selected from the group consisting of lactobionate, gluconate, citrate and glycerophosphate; and
(g) DMSO;
and wherein the second composition comprises albumin.

2-27. (canceled)

28. A cell culture of cells from an immortalized non-Hodgkin's lymphoma derived cytolytic cancer cell line (CRL-2407) comprising:
the cells and a cell freezing medium, wherein the cell freezing medium comprises a first composition and a second composition, wherein the first composition comprises:
(a) one or more electrolytes selected from the group consisting of potassium ions, sodium ions, magnesium ions, and calcium ions;
(b) one or more macromolecular agents selected from the group consisting of human albumin, polysaccharide and colloidal starch;
(c) a pH buffer;
(d) at least one sugar;
(e) at least one sugar alcohol;
(f) an impermeant agent being at least one member selected from the group consisting of lactobionate, gluconate, citrate and glycerophosphate; and
(g) DMSO; and
wherein the second composition comprises albumin.

29-44. (canceled)

45. A cell freezing medium produced by mixing a first composition and a second composition at a one to one ratio, wherein the first composition comprises:
(a) one or more electrolytes selected from the group consisting of potassium ions, sodium ions, magnesium ions, and calcium ions;
(b) one or more macromolecular agents selected from the group consisting of human albumin, polysaccharide and colloidal starch;
(c) a pH buffer;
(d) at least one sugar;
(e) at least one sugar alcohol;
(f) an impermeant agent selected from the group consisting of lactobionate, gluconate, citrate and glycerophosphate; and
(g) DMSO;
and wherein the second composition comprises albumin.

46. A method of harvesting cells from an immortalized non-Hodgkin's lymphoma derived cytolytic cancer cell line (CRL-2407) comprising collecting the cells from a cell culture and washing the collected cells by resuspending the cells in a buffer comprising 1-5% albumin.

47. The method of claim 46, wherein the collecting comprises centrifuging the cells in the cell culture.

48. The method of claim 46, further comprising placing the washed cells in an infusion bag.

49. The method of claim 46, wherein the wash is performed by centrifuging the cells and then resuspending the cells in the wash buffer.

50. The method of claim 46, wherein the wash is performed at least three times.

51. The method of claim 46, wherein the wash is performed 4-6 times.

52. The method of claim 51, wherein the method recovers at least 80% of the cells.

53. The method of claim 46, wherein viability of the harvested cells is at least 90%.

54. The method of claim 46, wherein the cells that have been harvested have substantially the same cytotoxicity and/or viability as (a) control cells that have not been harvested, or (b) cells before harvesting.

55. (canceled)

56. The method of claim 46, wherein the cells that have been harvested have a cytotoxicity of 80-100% on K562 cells.

57. The method of claim 46, wherein the buffer contains 2-5% albumin.

58. The method of claim 46, wherein the buffer contains 3-5% albumin.

59. (canceled)

60. The method of claim 46, wherein the buffer lacks sugar and/or dextran.

61. (canceled)

62. The method of claim 49, wherein the centrifugation is by continuous centrifugation.

63. The method of claim 46, wherein the albumin is human albumin, or human albumin derived from serum, or human albumin derived from plasma.

64. The method of claim 46, wherein the cells express a cytokine, Fc Receptor, a chimeric antigen receptor, or a combination thereof.

65. The method of claim 64, wherein the chimeric antigen receptor is a receptor for HER2, CD19, PD-L1, or a receptor for any of the tumor specific antigens listed in Table 2.

66. (canceled)
